(19) United States
(12) Patent Application Publication
(10) Pub. No.: US 2005/0014730 A1
(43) Pub. Date: Jan. 20, 2005

(54) Anti-fungal formulation of triterpene and essential oil

(76) Inventors: Robert M. Carlson, Duluth, MN (US); David J. Gibson, Woodbury, NJ (US)

Correspondence Address: Schwegman, Lundberg, Woessner & Kluth, P.A., P.O. Box 2938, Minneapolis, MN, 55402, US

(21) Appl. No.: 10/816,804

(22) Filed: Apr. 2, 2004

Related U.S. Application Data
(60) Provisional application No. 60/459,742, filed on Apr. 2, 2003.

(51) Int. Cl. A61K 31/56; A61K 35/78

(52) U.S. Cl. 514/169; 424/725

(57) ABSTRACT

The present invention provides for pharmaceutical compositions that includes a triterpene (e.g., betulin) and an essential oil (Vicks® Vapor Rub). The present invention also provides for a cosmetic formulation that includes a triterpene (e.g., betulin) and an essential oil (Vicks® Vapor Rub). The present invention also provides a method of treating a fungal infection that includes administering (e.g., topically applying) an effective amount of the pharmaceutical composition to the tissue afflicted with the fungal infection, or the tissue at risk of being afflicted with the fungal infection.

### RELATED APPLICATIONS

This application claims priority to U.S. Provisional Patent Application Ser. No. 60/459,742, filed Apr. 2, 2003, which is incorporated herein by reference.

### BACKGROUND OF THE INVENTION

Fungi infect humans and are a major cause of human health problems. They also infect plants and cause enormous losses in agricultural productivity. One class of fungal infections of mammals are the dermatophytic infections. These are fungal infections of the hair, nails, and skin. They are caused by fungi called “dermatophytes,” which include species belonging to the genera Epidermophyton, Microsporum, and Trichophyton. Among the species of dermatophytes are the following: Microsporum canis, which results in scalp and skin infections, mostly in children; Microsporum gypseum, which also results in scalp and skin infections in animals and humans; Trichophyton tonsurans, the major agent causing scalp ringworm; Trichophyton rubrum, causing skin, nail, hair, and scalp infections; and Trichophyton mentagrophytes, which can occur on all parts of the body surface. Other fungal infectious agents include the opportunists that are likely to infect immunodeficient persons. These include Cryptococcus, Candida, and Aspergillus.

Outer layers of plants such as leaf cuticles, fruit peels, and bark protect the plant against abrasion, prevent water loss, and protect against pathogenic microorganisms. Breaking through the plant protective outer layer is a prerequisite for a pathogen to enter the plant's internal tissues. Some studies have suggested that penetration of the protective layer involves dissolution of the host cuticle by enzymes secreted by the pathogen. Nicholson, R. L. et al., in The Fungal Spore and Disease Initiation in Plants and Animals, eds. Cole, G. T., and Hoch, H. C., 1991, Plenum Press, New York, pp. 3-23.

Pentacyclic triterpenes are among the most common plant secondary metabolites, but their function in plants has not been fully understood. They are usually concentrated in the outermost layers such as plant cuticle, fruit peel, and bark.

Literature supplies examples of enzymes that can be inhibited by triterpenes, indicating the ability of triterpenes to act broadly in a non-specific mode on multiple targets. For example, Buchler et al. (Biochem. Biophys. Acta 1075, 206 (1991) showed inhibition of rat renal 11β-hydroxysteroid dehydrogenase. Koch et al. (Phytother, Res. 8, 109 (1994)) showed in vitro inhibition of adenosine deaminase. This leads to the hypothesis that pentacyclic triterpenoids in plant protective outer layers may protect against infection by inhibiting enzymes that would degrade the cuticle.

Betulin is a pentacyclic triterpenoid derived from the outer bark of paper birch trees (Betula papyrifera, B. pendula, B. verucosa, etc.). It can be present at concentrations of up to about 24% of the bark of white birch. Merck Index, twelfth edition, page 1236 (1996). Lupeol is a related compound also found in birch bark and in other plant sources. Lupeol is present at concentrations of about 1.5-3% of the birch bark and at up to about 8.2% in Canavalia ensiformis, a plant widespread in the humid tropics of Asia and Africa. Allobetulin is another triterpenoid found in birch bark. A typical pulp mill that process birch produces enough bark waste to allow for the inexpensive isolation of significant quantities of these triterpenoids.

Several triterpenoids have been found to have utility. For example, betulin and related compounds have been shown to have anti-viral activity against herpes simplex virus. Carlson et al., U.S. Pat. No. 5,750,578. Betulin and related compounds have also been shown to have anti-fungal and anti-bacterial activity. However, triterpenoids are hydrophobic compounds with relatively low interfacial activity and water solubility. For instance, the solubility of betulin in water is about 0.15 mg/l. The relatively low interfacial activity and water solubility can make handling and administration of the compounds difficult. Low interfacial activity also limits the efficient interaction with target (fungi or bacteria) cell membranes. It also limits accessibility to hydrophilic biological targets or targets protected by a hydrophilic barrier.

Current agents used to treat fungal infections include the polyene antibiotics, including nystatin; synthetic azoles; and griseofulvin. Fungal infections are difficult to treat because, like humans, they are eukaryotes.

Although many triterpenes have biological activity, the use of triterpenes, particularly for treating plants, presents several drawbacks. Triterpenes dissolve sparingly in water and other aqueous media and thus are difficult to apply to crops in non-emulsion formulations.

Currently, there is a need for new anti-fungal compositions that include triterpenes. The new anti-fungal compositions would include a triterpene in a carrier that could effectively dissolve an effective and safe amount of the triterpene. A need particularly exists for compositions that will act against a range of species, including dermatophytic fungi. New anti-fungal compositions would be less expensive to manufacture if they were abundant natural products or easily synthesized from abundant natural products. As such, the compositions would have biological activity against a range of species, including dermatophytic fungi.

### SUMMARY OF THE INVENTION

The present invention provides for new anti-fungal compositions that include triterpenes. The new anti-fungal compositions include a triterpene in a carrier that effectively dissolves an effective and safe amount of the triterpene. The compositions act against a range of species, including dermatophytic fungi. The anti-fungal compositions are less expensive to manufacture or include triterpenes that are easily synthesized from abundant natural products. As such, the compositions would have biological activity against a range of species, including dermatophytic fungi.

The present invention provides a pharmaceutical composition that includes a triterpene and an essential oil.

The present invention provides a cosmetic composition that includes a triterpene and an essential oil.

The present invention also provides an anti-fungicidal composition that includes a composition of the present invention and a fungicidal excipient.

The present invention also provides a therapeutic method for treating a mammal afflicted with a fungal infection that includes administering to the mammal, an effective anti-fungal amount of a composition of the present invention.

The present invention also provides a cosmetic method for alleviating the physical symptoms associated with a mammalian fungal infection, that includes administering to the mammal, an effective anti-fungal amount of a composition of the present invention.

The present invention also provides a method of inhibiting or killing a fungus that includes contacting the fungus with an effective anti-fungal amount of a composition of the present invention.

### DETAILED DESCRIPTION OF THE INVENTION

The following definitions are used, unless otherwise described: halo is fluoro, chloro, bromo, or iodo. Alkyl, alkoxy, alkenyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic.

It will be appreciated by those skilled in the art that triterpene compounds present in the compositions of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound present in the compositions of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antifungal activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl;
partially unsaturated (C₂-C₆)alkyl or (C₂-C₆)alkenyl can be vinyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl;
(C₁-C₅)alkanoyl can be carbonyl, acetyl, propanoyl, butanoyl, isopropanoyl, or pentenoyl;
(C₁-C₆)alkoxy can be methoxy, ethoxy, propoxy, isopropoxy, butoxy, iso-butoxy, sec-butoxy, pentoxy, 2-pentoxy, 3-pentoxy, or hexyloxy;
halo(C₁-C₆)alkoxy can be trifluoromethyloxy, 2-chloroethyloxy, 3,3-dichloropropyloxy, or 4,4,4-trifluorobutyloxy;
(C₃-C₈)cycloalkyl can be cyclopropyl, cyclobutyl, cyclopentyl, cyclohexyl, cycloheptyl, or cyclooctyl;
(C₃-C₈)cycloalkyloxy can be cyclopropyloxy, cyclobutyloxy, cyclopentyloxy, cyclohexyloxy, cycloheptyloxy, or cyclooctyloxy;
hydroxy(C₁-C₆)alkoxy can be hydroxymethoxy, 1-hydroxyethoxy, 2-hydroxyethoxy, 1-hydroxypropoxy, 2-hydroxypropoxy, 3-hydroxypropoxy, 1-hydroxybutoxy, 4-hydroxybutoxy, 1-hydroxypentoxy, 5-hydroxypentoxy, 1-hydroxyhexoxy, or 6-hydroxyhexoxy;
amino(C₁-C₆)alkyl can be aminomethyl, 1-aminoethyl, 2-aminoethyl, 1-aminopropyl, 2-aminopropyl, 3-aminopropyl, 1-aminobutyl, 2-aminobutyl, 3-aminobutyl, 4-aminobutyl, 1-aminopentyl, 2-aminopentyl, 3-aminopentyl, 5-aminopentyl, 1-aminohexyl, 2-aminohexyl, 3-aminohexyl, or 6-aminohexyl;
(C₁-C₆)alkoxycarbonyl can be methoxycarbonyl, ethoxycarbonyl, propyloxycarbonyl, isopropyloxycarbonyl, 2-methylpropyloxycarbonyl, butyloxycarbonyl, pentyloxycarbonyl, or hexyloxycarbonyl;
(C₁-C₆)alkanoyloxy can be carbonyloxy, acetyloxy, propanoyloxy, butanoyloxy, 2-methylpropanoyloxy, 2-methylbutanoyloxy, 3-methylbutanoyloxy, pentanoyloxy, or hexanoyloxy.

“N⁺-containing heteroaryl” can be N-pyridinium, N-methyl-2-pyridinium, N-methyl-3-pyridinium, N-methyl-4-pyridinium, N-ethyl-2-pyridinium, N-ethyl-3-pyridinium, N-ethyl-4-pyridinium, 3,5-dimethylpyridinium, or 4-(dimethylamino)pyridinium.

“N⁺-containing heterocycle” can be N-diazabicyclo[2.2.2]octyl; N-azabicyclo[2.2.2]octyl; N-methyl-N-piperidino; N,N-dimethyl-2-piperidino; N,N-dimethyl-3-piperidino; N,N-dimethyl-4-piperidno; N-methyl-N-morpholino; N,N-dimethyl-2-morpholino; or N,N-dimethyl-3-morpholino.

“—N⁺—RₐR_{b}R_{c}” can be N′-benzyl-N,N,N′,N′-tetramethylethylenediamine-N-yl; N,N,N′,N′-tetramethylethylenediamine-N-yl; octyldimethylammonium; tetradecyldimethylammonium; trimethylammonium; triethylammonium, or tri(hydroxymethyl)ammonium.

“3-Carboxypropenoyloxymethyl” refers to the structure
—CH₂OC(═O)CH═CHCOOH.

“Aminoacetoxymethyl” refers to the structure —CH₂C(═O)CH₂NH₂.

“(Carboxymethoxy)acetoxymethyl” refers to the structure
—CH₂OC(═O)CH₂OCH₂COOH.

“4-Carboxybutanoyloxymethyl” refers to the structure
—CH₂OC(═O)CH₂CH₂CH₂COOH.

“3-Carboxypropanoyloxymethyl” refers to the structure
—CH₂OC(═O)CH₂CH₂COOH.

“Carboxycarbonyloxymethyl” refers to the structure
—CH₂OC(═O)COOH.

“2-Amino-3-methyl-butanoyloxymethyl” refers to the structure
—CH₂OC(═O)CH(NH₂)CH(CH₃)₂.

“4-Carboxy-(3,3-dimethyl)butanoyloxymethyl” refers to the structure —CH₂C(═O)CH₂C(CH₃)₂CH₂COOH.

“2-Carboxybenzoyloxymethyl” refers to the structure

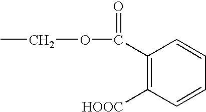

“Butanoyloxymethyl” refers to the structure —CH₂OC(═O)CH₂CH₂CH₃.

“2-Carboxybenzoyl” refers to the structure

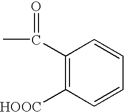

“2-Amino-3-methylbutanoyl” refers to the structure —C(═O)CH₂(NH₂)CH₂(CH₃)₂.

“3-Carboxypropenoyl” refers to the structure —C(═O)CH═CHCOOH.

“Aminoacetyl” refers to the structure —C(═O)CH₂NH₂.

“4-Carboxybutanoyl” refers to the structure —C(═O)CH₂CH₂CH₂COOH.

“(Carboxymethoxy)acetyl” refers to the structure —C(═O)CH₂OCH₂COOH.

“3-(3,4-Dihydroxyphenyl)propenoyl” refers to the structure

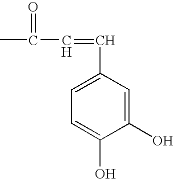

“3-Carboxypropanoyl” refers to the structure —C(═O)CH₂CH₂COOH.

“Carboxycarbonyl” refers to the structure —C(═O)COOH.

“4-Carboxy-(3,3-dimethyl)butanoyl” refers to the structure
—C(═O)CH₂C(CH₃)₂CH₂COOH.

“Carboxymethylenethioacetyl” refers to the structure —C(═O)CH₂SCH₂COOH.

“3-Carboxy-3-methylbutanoyl” refers to the structure —C(═O)CH₂C(COOH)(CH₃)₂.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Third Edition, Wiley: New York, 1999, and references cited therein). An amino acid can be linked to the remainder of a compound of formula (I)-(VI) through the carboxy terminus, the amino terminus, or through any other convenient point of attachment, such as, for example, through the sulfur of cysteine.

The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula (I)-(VI) through the carboxy terminus, the amino terminus, or through any other convenient point of attachment, such as, for example, through the sulfur of a cysteine. Preferably a peptide comprises 3 to 25, or 5 to 21 amino acids. Peptide derivatives can be prepared as disclosed in U.S. Pat. Nos. 4,612,302; 4,853,371; and 4,684,620.

Glycosides are formed by reacting mono-, di- and polysaccharides with 1-2 hydroxyl groups of the compound of formula (I)-(VI), including glucose, glucuronic acid, mannose, galactose, sorbase, ribose, maltose, sucrose, modified cellulosics, dextrans, modified starches and the like. These derivatives can advantageously exhibit improved water solubility over betulin itself. See, Remington's Pharmaceutical Sciences, A. R. Gennaro, ed., Mack Pub. Co. (18th ed., 1990) at pages 384-386. Glycoside derivatives can be prepared as described in PCT Applications WO 96/34005 and 97/03995.

The term “polyethyleneimine” refers to the group (—NHCH₂CH₂—)ₓ[—N(CH₂CH₂NH₂)CH₂CH₂—]_{y}. Polyethyleneimine can be attached to a compound through either of the nitrogen atoms marked with hash marks. “Poly(ethylene glycol)” refers to the compound H(OCH₂CH₂)ₙOH. It can be attached to a compound through its terminal hydroxyl.

The term “partially unsaturated” refers to a linear or branched hydrocarbon having one or more carbon-carbon double bonds.

The term “phosphono” refers to O═P(OH)₂—.

The term “direct bond” refers to a group being absent.

Combinations of substituents and/or variables are permissible only if such combinations result in stable compounds. By “stable compound” is meant herein a compound that is sufficiently robust to survive isolation to a useful degree of purity from a reaction mixture, and formulation into an efficacious antifungal agent.

As used herein, the term “triterpene” can be a plant secondary metabolite that includes a hydrocarbon, or its oxygenated analog, that is derived from squalene by a sequence of straightforward cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd. Ed. (London: Chapman 1989). The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. Examples of triterpenes include betulin, allobetulin, lupeol, friedelin, and all sterols, including lanosterol, stigmasterol, cholesterol, β-sitosterol, and ergosterol.

The term, “essential oil” refers to a highly odoriferous, volatile liquid component obtained from plant tissue. Essential oils typically include a mixture of one or more terpenes, esters, aldehydes, ketones, alcohols, phenols, and/or oxides. These functional classes of compounds are responsible for the therapeutic properties and distinct fragrance of the essential oil.

The essential oil can be manufactured (i.e., synthesized or partially synthesized). Alternatively, the essential oil can be obtained from a plant or plant component (e.g., plant tissue). Suitable plant or plant components include, e.g., a herb, flower, fruit, seed, bark, stem, root, needle, bulb, berry, rhizome, rootstock, leaf, or a combination thereof.

Any suitable essential oil can be employed provided (1) the essential oil has therapeutic properties (e.g., the essential oil has anti-fungal properties), (2) the essential oil provides a scent that is associated with plant tissue, (3) the essential oil remains stable in the composition, and/or the essential oil at least partially dissolves the triterpene. Preferably, the stability is over a prolonged period of time, e.g., up to about 3 years, up to about 1 year, or up to about 6 months, typically experienced in the manufacturing, packaging, shipping, and/or storage of the composition. The specific essential oil will preferably be non-toxic to mammals (e.g., humans) and will be suitable for medicinal use (e.g., topically). The specific essential oil will also preferably comply with any controlling or governing body of law, e.g., FDA regulations.

Suitable specific essential oils include, e.g., one or more of the following: ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenugreek, fir, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, Indian patchouli oil, Indonesian patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra.

Specifically, suitable exemplary essential oils include, e.g., angelica root, anise, basil (e.g., sweet French basil), bay leaf, benzoin absolute, bergamot, birch, carrot seed, cedarwood, chamomile (e.g., German chamomile, Moroccan chamomile, or Roman chamomile), cinnamon leaf, cinnamon cassia, cistus, citronella, clary sage, clove bud, cypress, eucalyptus globulus, eucalyptus citriodora, everlasting (helicrysum), fennel, fir, frankincense, geranium, ginger, grapefruit, helichrysum, hyssop, juniper berry, lavender, lavendin, lemon, lemongrass, lime, marjoram, myrrh, myrtle, neroli, niaouli, nutmeg, sweet orange, oregano, patchouli, pennyroyal, peppermint, petitgrain, pepper, pine needle, ravensare, rose geranium, rosemary (e.g., Spanish rosemary), rosewood, sage, sandalwood, spikenard, spruce, tangerine, tarragon, tea tree, thyme, vanilla, vetiver, ylang ylang, or a combination thereof.

In one specific embodiment of the present invention, the essential oil can include, e.g., the combination of menthol, camphor, eucalyptus oil, cedarleaf oil, nutmeg oil, thymol, and turpentine oil. In another specific embodiment of the present invention, the essential oil can exclude, e.g., the combination of menthol, camphor, eucalyptus oil, cedarleaf oil, nutmeg oil, thymol, and turpentine oil.

In one specific embodiment of the present invention, the essential oil includes Vicks® Vapor Rub. It has surprisingly been discovered that Vicks® Vapor Rub effectively solubilizes an effective anti-fungal amount of a triterpene (e.g., betulin), while maintaining the stability and anti-fungal activity of the triterpene.

Other suitable essential oils that can be employed in the compositions of the present invention are disclosed in the following websites: www.essential-essences.com; www.fragrancefactory.com; www.essentialoil.com; www.essentialoils.org; www.halcyon.com; and www.essential-oil.org; which are all incorporated by reference herein.

The term “quaternary ammonium salt” refers to a compound comprising at least one positively charged nitrogen atom with four covalent bonds to non-hydrogen atoms. Typically the four bonds will be to carbon atoms. Two or three of the bonds can make up a double or triple bond respectively to a single atom.

The triterpenes present in the compositions of the instant invention also include triterpenes derivatized with N⁺-containing groups. These compounds are found to be rather resistant to hydrolysis. Derivatization with N⁺-containing groups is also found to make the triterpenes present in the compositions of the instant invention rather water soluble. For instance, the solubility of some quaternary salts of betulin disclosed herein is 400-600 g/l.

The term “quaternary ammonium salt of a triterpene” refers to triterpene covalently attached to a group comprising at least one positively charged nitrogen atom with four covalent bonds to non-hydrogen atoms. Examples of quaternary ammonium salts of a triterpene include a compound of formulas (I)-(IV).

The term “fungus” refers to a distinct group of eukaryotic, spore-forming organisms wih absorptive nutrition and lacking chlorophyll. It includes mushrooms, molds, and yeasts.

The term “N-diazabicyclo[2.2.2]octyl” refers to the group

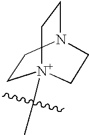

The term “N-pyridinium” refers to the group

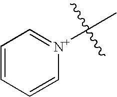

The term “N-methyl-N-piperidino” refers to the group

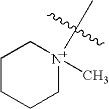

The term “N-methyl-N-morpholino” refers to the group

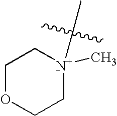

The term “N-azabicyclo[2.2.2]octyl” refers to the group

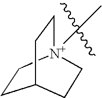

The structure and carbon numbering of three exemplary compounds present in the compositions of the 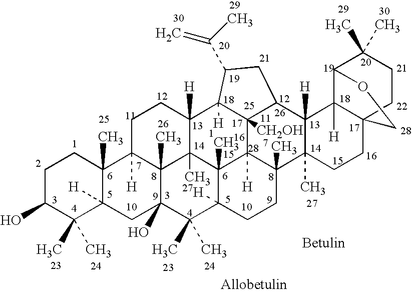
instant invention are shown below.

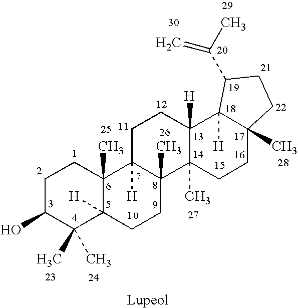

Specific values for compounds of formula (I) are as follows.

A specific value for the bond between carbons 1 and 2 is a single bond.

Another specific value for the bond between carbons 1 and 2 is a double bond.

A specific value for R₁ is hydrogen.

Another specific value for R₁ is hydroxy.

A specific value for R₂ is a direct bond.

A specific value for R₃ is (C₁-C₆)alkyl; wherein any alkyl can optionally be substituted with one or more oxo, carboxy, amino, —OP(═O)(OH)₂, or phenyl; any alkyl is optionally interrupted on carbon with one or more oxy or thio; any alkyl is optionally partially unsaturated; and any aryl can optionally be substituted with one or more hydroxy or carboxy.

Another specific value for R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂OC(═O)C(═O)—(—NHCH₂CH₂)ₓ—[—N(CH₂CH₂NH₂)CH₂CH₂]_{y}.

A specific value for R₄ is hydrogen or (C₁-C₆)alkyl; wherein any alkyl can optionally be substituted with one or more oxo, carboxy, amino, —OP(═O)(OH)₂, or phenyl; any alkyl is optionally interrupted on carbon with one or more oxy or thio; any alkyl is optionally partially unsaturated; and any aryl can optionally be substituted with one or more hydroxy or carboxy.

Another specific value for R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)ₓ—[—N(CH₂CH₂NH₂)CH₂CH₂]_{y}.

A specific value for R₅ is oxy.

A specific group of compounds are compounds of formula (I) wherein R₁ is hydrogen or hydroxy; R₂ is a direct bond; R₃ is (C₁-C₆)alkyl; R₄ is hydrogen or (C₁-C₆)alkyl; and R₅ is oxy or R₄ and R₅ together are oxo; wherein any alkyl can optionally be substituted with one or more oxo, carboxy, amino, or —OP(═O)(OH)₂, or phenyl; any alkyl is optionally interrupted on carbon with one or more oxy or thio; any alkyl is optionally partially unsaturated; and any aryl can optionally be substituted with one or more hydroxy or carboxy.

Another specific group of compounds are compounds of formula (I) wherein R₁ is hydrogen or hydroxy; R₂ is a direct bond; R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂C(═O)C(═O)—(—NHCH₂CH₂)ₓ—[—N(CH₂CH₂NH₂)CH₂CH₂]_{y}; R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)ₓ—[—N(CH₂CH₂NH₂)CH₂CH₂]_{y}.; and R₅ is oxy or R₄ and R₅ together are oxo.

Another specific group of compounds of formula (I) is betulin; betulin-3,28-diglycine; betulin-28-glycerol oxalate; betulin-28-glycine; betulin-28-oxalate; betulin arabinose galactan; betulin-3,28-diglycolate; betulin-3-maleate; betulin-3,28-di-(L-glutamic acid γ-benzylester) ester; betulin-3,28-di-L-alanine; betulin-3,28-di-L-proline ester; betulin-3,28-dioxalate; betulin-1-ene-2-ol; betulin-3,28-diphenylalanine; betulin-3,28-di-(L-proline ester); betulin-3,28-dioxalate-polyethylene amine; betulin-3,28-diphosphate; betulin-3-caffeate; betulin-3,28-(3′,3′-dimethyl)glutarate; betulin-28-diglycolate; betulin-28-glutarate; betulin-28-maleate; betulin-28-phthalate; betulin-3,28-di(31,3′-dimethyl) glutarate; betulin-3,28-didiglycolate; betulin-3,28-dithiodiglycolate; betulin-3,28-diglutarate; betulin-3,28-dimaleate; betulin-3,28-diglycolate; betulin-3,28-diphthalate; betulin-3,28-di-L-valine ester; betulin-28-succinate; betulin-3,28-disuccinate; betulin-3,28-di-(polyethylene glycol)-COOH (Mw=1448); betulin-3,28-di-(polyethylene glycol)-COOH (Mw=906 crude); betulin-3,28-di-(polyethylene glycol)-COOH (Mw=906 pure); betulinic acid; betulon-1-ene-2-ol; betulin-3,28-(dipoly(ethylene glycol)bis(carboxymethylester); hederin hydrate; lupeol; lupeol-3-glutarate; lupeol-3-succinate; lupeol-3-thiodiglycolate; lupeol-3-phthalate; oleanolic acid; ursolic acid; or uvaol.

Another specific group of compounds of formula (I) is betulin; betulin-3,28-diglycine; betulin-28-glycerol oxalate; betulin-28-glycine; betulin oxalate; betulin arabinose galactan; betulin-3,28-diglycolate; betulin-3-maleate; betulin di-(L-glutamic acid γ-benzylester) ester; betulin 3,28-di-L-alanine; betulin 3,28-di-L-proline; betulin-3,28-dioxalate; betulin-1-ene-2-ol; betulin-3,28-diphenylalanine ester; betulin-3,28-dioxalate-(polyethylene amine); betulin-3-caffeate; betulin-3,28-(3′,3′-dimethyl)glutarate; betulin-28-diglycolate; betulin-28-glutarate; betulin-28-phthalate; betulin-3,28-diglycolate; betulin-3,28-diphthalate; betulin-3,28-phosphate; betulin-28-succinate; betulin-3,28-disuccinate; betulin-3,28-di-(polyethylene glycol)-COOH (Mw=1448); betulin-3,28-di-(polyethylene glycol)-COOH (Mw=906 crude); betulin-3,28-di-(polyethylene glycol)-COOH (Mw=906 pure); betulon-1-ene-2-ol; betulin-3,28-(dipoly(ethylene glycol)bis(carboxymethylester); hederin hydrate; lupeol-3-succinate; lupeol-3-phthalate; lupeol-3-glutarate; oleanolic acid; ursolic acid; or uvaol.

Another specific group of compounds of formula (I) is betulin; betulin-3-maleate; betulin-28-diglycolate; betulin-28-glutarate; betulin-28-maleate; betulin-28-phthalate; betulin-28-succinate; betulin-3,28-diglycine; betulin-3,28-didiglycolate; betulin-3,28-dimaleate; betuliii-3,28-dioxalate-3-polyethyleneimine; betulin-3,28-di(3′,3′-dimethyl)glutarate; betulin-3,28-dioxalate-3,28-polyethyleneimine; betulin-3,28-diphthalate; betulin-3,28-disuccinate; betulin-3,28-di-L-valine; lupeol; lupeol-3-amine; lupeol-3-(3′,3′-dimethyl)succinate; lupeol-3-maleate; lupenone; or lupenon-1,2-ene-2-ol.

Specific values for the compounds of formula (II) are as follows.

A specific value for the bond between carbons 1 and 2 is a single bond.

A specific value for R₁ is —O—Y, wherein Y is hydrogen, an amino acid, or (C₁-C₆)alkyl; wherein any alkyl can be optionally substituted with one or more oxo, hydroxy, amino, phenyl, or carboxy any alky can be optionally interrupted with one or more oxy or thio; any phenyl can be optionally substituted with one or more hydroxy or carboxy.

Another specific value for R₁ is —O—Y, wherein Y is hydrogen, 3-carboxypropanoyl, 4-carboxybutanoyl, or 2-amino-2-methylbutanoyl.

A specific value for R₂ is hydrogen.

A specific value for R₃ is hydrogen.

A specific value for R₄ is methyl.

A specific value for R₅ is methyl.

A specific value for R₆ is hydrogen.

A specific value for the bond between carbons 12 and 13 is a single bond.

A specific value for R₇ is hydrogen.

A specific value for R₈ and R₁₁ together is —O—CH₂—.

A specific value for R₉ is methyl.

A specific value for R₁₀ is methyl.

A specific group of compounds of formula (II) is the compounds wherein R₁ is —O—Y and Y is hydrogen, an amino acid, or (C₁-C₆)alkyl; wherein the alkyl of Y can be optionally substituted with one or more oxo, hydroxy, amino, carboxy, or phenyl optionally substituted with one or more hydroxy or carboxy; and can be optionally interrupted with one or more oxy or thio; R₂ is hydrogen; R₃ is hydrogen and the bond between carbons 1 and 2 is a single bond; R₄ and R₅ are each methyl; R₆ is hydrogen and the bond between carbons 12 and 13 is a single bond; R₇ is hydrogen; R₈ and R₁₁ together are —O—CH₂—; and R₉ and R₁₀ are each methyl.

Another specific group of compounds of formula (II) is 3-β-acetoxy-19αH-19,28 lactone oleanan; allobetulin; allobetulin-3-succinate; allobetulin-3-glycine; allobetulin lactone; allobetulin lactone-3-acetate; allobetulin lactone-3-phosphate; allobetulin-3-L-alanine; allobetulin-3-L-valine; allobetulin-3-L-proline ester; allobetulin-3-succinate; allobetulin-3-diglycolate; allobetulin-3-phthalate; allobetulin-3-methylenamine; allobetulin-3-ethanolamine; allobetulin-3-glycolate; allobetulin-3-glutarate; allobetulin-28-glutarate; allobetulin-3-methylamine HCl; allobetulin-3-phosphate; allobetulin-3-(polyethylene glycol)-COOH (Mw=674); allobetulon; allobetulon lactone-1-ene-2-ol; allobetulon lactone-1-en-2-succinate; allobetulon-1-ene-2-ol; allobetulon-1-ene-2-diglycolate; 3-allobetulon-1-ene-2-succinate; allobetulin-3-(poly(ethylene glycol)bis (carboxymethyl ester); or 3-allobetulon-1-ene-2-diglycolate.

Another specific group of compounds of formula (II) is 3-β-acetoxy-19αH-19,28 lactone oleanan; allobetulin; allobetulin-3-succinate; allobetulin lactone; allobetulin lactone-3-acetate; allobetulin lactone-3-phosphate; allobetulin-3-L-valine; allobetulin-3-L-proline; allobetulin-3-succinate; allobetulin-3-diglycolate; allobetulin-3-methylenamine; allobetulin-3-ethanolamine; allobetulin-3-glycolate; allobetulin-3-glutarate; allobetulin-3-glutarate; allobetulin-3-(polyethylene glycol)-COOH (Mw=674); allobetulon; allobetulon lactone-1-ene-2-ol; allobetulon lactone-1-en-2-succinate; allobetulon-1-ene-2-ol; allobetulon-1-ene-2-diglycolate; 3-allobetulon-1-ene-2-succinate; or allobetulin-3-(poly(ethylene glycol)bis(carboxymethyl ester).

Another specific group of compounds of formula (II) is allobetulin, allobetulin-3-glutarate, allobetulin-3-succinate, or allobetulin-3-L-valine.

In one specific embodiment of a compound of formula (IV), R₂, R₅, and R₈ are each independently absent, hydroxyl, N-diazabicyclo[2.2.2]octyl, N-pyridinium, N-alkyl-N-piperidino, N-alkyl-N-morpholino, N-azabicyclo[2.2.2]octyl, or NRₐR_{b}R_{c}; provided at least one of R₂, R₅, and R₈ is N⁺-containing heteroaryl, N⁺-containing heterocycle, or —N⁺RₐR_{b}R_{c}. In this embodiment N-diazabicyclo[2.2.2]octyl; N-pyridinium; N-alkyl-N-piperidino; N-alkyl-N-morpholino; and N-azabicyclo[2.2.2]octyl can optionally be substituted on one or more suitable carbon atoms with one or more oxo, hydroxy, mercapto, alkyl, hydroxyalkyl, halo, nitro, cyano, (C₁-C₆)alkoxy, —COOR_{d}, or —NR_{d}Rₑ. In this embodiment also, any alkyl or alkylene of R₁, R₂, R₄, R₅, R₇, or R₈ can optionally be substituted with one or more oxo or —NR_{d}Rₑ, and optionally interrupted with one or more oxy, imino, or thio, and can optionally be partially unsaturated.

In another specific embodiment of a compound of formula (IV), R₁ is absent and R₂ is hydrogen, N-diazabicyclo[2.2.2]octyl, or N-dimethylamino-N-pyridinium.

In another specific embodiment of a compound of formula (IV), R₃ and R₄ are absent, and R₅ is hydrogen.

In another specific embodiment of a compound of formula (IV), R₃ is oxy; R₄ is absent or (C₁-C₅)alkylenecarbonyl; and R₅ is hydrogen, N-diazabicyclo[2.2.2]octyl; 4-dimethylamino-N-pyridinium; 4-hydroxybutyl-N-diazabicyclo[2.2.2]octyl; 4-benzyl-N-diazabicyclo[2.2.2]octyl; tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; or tetradecyldimethylammonium.

In another specific embodiment of a compound of formula (IV), R₆ is oxy; R₇ is absent or (C₁-C₅)alkylenecarbonyl; and R₈ is hydrogen, N-diazabicyclo[2.2.2]octyl; 4-dimethylamino-N-pyridinium; N′-(4-hydroxybutyl)-N-diazabicyclo[2.2.2]octyl; N′-benzyl-N-diazabicyclo[2.2.2]octyl; N,N,N′,N′-tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; tetradecyldimethylammonium; 2-methyl-N-pyridinium; 4-hydroxy-N-methyl-N-piperidinium; or N-methyl-N-morpholino.

In particular embodiments of the invention, the compound of formula (IV) is:
lup-20(29)-ene-3,28-bis-(N-pyridiniumacetate);
lup-20(29)-ene-3-[N-(4-oxybutyl)-1,4-diazabicyclo[2.2.2]octyl-N′-acetate];
lup-20(29)-ene-3,28-bis[N-(1,4-diazabicyclo[2.2.2]octyl)acetate];
lup-20(29)-ene-3,28-bis[N—(N′-benzyldiazabicyclo[2.2.2]octyl)acetate);
lup-20(29)-ene-3,28-bis[N—(N′-(4-oxybutyl)diazabicyclo[2.2.2]octyl)acetate];
lup-20(29)-ene-3-[N-(1,4-diazabicyclo[2.2.2]octyl)acetate];
lup-20 (29)-ene-3,28-bis[(tetramethyletylenediamine-N-yl)acetate];
lup-20(29)-ene-3,28-bis[N′-benzyl-N,N,N′,N′-tetramethylethylenediamine-N-yl)acetate];
lup-20(29)-ene-3-[N-(N′-(benzyl)diazabicyclo[2.2.2]octyl)acetate];
bis(N,N′-pyridinium-2-ethyl)lup-20(29)-ene-3,28-dicarbamate;
1-(3,28-(diacetoxy)lup-20(29)-ene-30-yl)-4-(dimethylamino)pyridinium;
lup-20(29)-ene-3,28-bis(N-pyridinium-2-propionate); lup-20(29)-ene-3,28-bis(N-pyridinium-3-propionate);
lup-20(29)-ene-3,28-bis(N-pyridinium-4-butyrate);
lup-20(29)-ene-3,28-bis(N-pyridinium-4-butyrate); lup-20(29)-ene-3,28-bis(N-pyridinium-2-butyrate);
1-[3,28-(diacetoxy)lup-20(29)-ene-30-yl]-1,4-diazabicyclo[2.2.2]octyl;
3,28-bis[3-(1-piperidinyl)propanoyloxy]lup-20(29)-ene;
1-(3,28-dihydroxylup-20(29)ene-30-yl)-4-(dimethylamino)pyridinium;
lup-20(29)-ene-3,28-bis[N-(4-dimethylaminopyridinium)-2-propionate];
lup-20(29)-ene-3,28-bis[N-(1,4-diazabicyclo[2.2.2]octyl)-2-propionate];
1-(lup-20(29)-ene-30-yl)-1,4-diazabicyclo[2.2.2]octane;
1-(3,28-dihydroxylup-20(29)-ene-30-yl)-pyridinium;
lup-20(29)-ene-3,28-bis[N-(1,4-diazabicyclo[2.2.2]octyl)-4-butyrate];
1-(3,28-dihydroxylup-20 (29)-ene-30-yl)-[N-3-(hydroxymethyl)pyridinium];
1-(3,28-dihydroxylup-20(29)-ene-30-yl)-[N-(3,5-dimethylpyridinium)];
bis[N-(1,4-diazabicyclo[2.2.2]octyl)-2-ethyl]-lup-20(29)ene-3,28-dicarbamate;
lup-20(29)-ene-3,28-bis[N-(3-oxymethylpyridinium)acetate];
lup-20(29)-ene-3,28-bis[N-(2-oxymethylpyridinium)acetate];
lup-20(29)-ene-3,28-bis[N-(2-methylureapyridinium)acetate];
lup-20(29)-ene-3-[N-(2-oxymethylpyridinium)acetate];
lup-20(29)-ene-3,28-bis[N-(N-methylmorpholino)acetate];
lup-20(29)-ene-3,28-bis[N-(4-hydroxyl-N-methylpiperidino)acetate];
lup-20(29)-ene-3-[N-(3-ureamethylpyridinium)acetate];
lup-20(29)-ene-3-(N-pyridiniumacetate);
lup-20(29)-ene-3,28-bis[N-(1,4-diazabicyclo[2.2.2]octyl)-2-butyrate];
lup-20(29)-ene-3,28-bis[N-(4-dimethylpyridinium)-2-butyrate];
lup-20(29)-ene-3,28-bis[N-(4-dimethylaminopyridinium)-4-butyrate];
lup-20(29)-ene-3,28-bis[N-(4-dimethylaminopyridinium)-3-propionate];
1-(3,28-dihydroxylup-20(29)-ene-30-yl)-4-(hydroxymethyl)pyridinium;
1-(3,28-dihydroxylup-20(29)-ene-30-yl)-3-hydroxy-1-azabicyclo[2.2.2]octane;
lup-20(29)-ene-3,28-bis[N-(2,4-dimethylpyridinium)acetate];
lup-20(29)-ene-3,28-bis[N-(3,5-dimethylpyridinium)acetate];
lup-20(29)-ene-3,28-bis[N-(4-dimethylaminopyridinium)acetate];
lup-20(29)-ene-3-[N-(2-methylpyridinium)acetate];
lup-20(29)ene-3-[N-(2,4-dimethylpyridinium)acetate];
lup-20(29)-ene-3-[N-(4-hydroxy-N-methylpiperidino)acetate];
lup-20(29)-ene-3-[N-(N-methylmorpholino)acetate];
lup-20(29)-ene-3-[N-(3,5-dimethylpyridinium)acetate];
lup-20(29)-ene-3-[N-(4-dimethylaminopyridinium)acetate];
lup-20(29)-ene-3,28-bis(octyldimethylammoniumacetate);
lup-20(29)-ene-3-octyldimethylammoniumacetate;
lup-20(29)-ene-3,28-bis(tetradecyldimethylammoniumacetate);
lup-20(29)-ene-3-tetradecyldimethylammoniumacetate;
N,N,N′,N′-tetramethylethylenediamine-N,N′-bis-[lup-20(29)-ene-3-acetate];
1-[(lup-20(29)-en-3β-yl)oxycarbonylmethyl]-4-aza-1-azonia-bicyclo[2.2.2]octane;
1-[(lup-20 (29)-en-3β-yl)oxycarbonylmethyl]trimethylammonium; or
1-[(lup-20(29)-en-3β-yl)oxycarbonylmethyl]pyridinium.

A specific embodiment of the compound of formula (VI) is the compound wherein R₁ is hydrogen, alkyl, or hydroxyalkyl; R₂ is oxymethylene, thiomethylene, iminomethylene, or methylene; R₃ and R₆ are each independently absent or alkylenecarbonyl; R₄ and R₇ are each independently hydrogen, N-diazabicyclo[2.2.2]octyl; N-pyridinium; N-alkyl-N-piperidino; N-alkyl-N-morpholino; N-azabicyclo[2.2.2]octyl; or NRₐR_{b}R_{c}; or R₁, R₂, R₃, and R₄ are together —C—CH₂—. In this case, N-diazabicyclo[2.2.2]octyl; N-pyridinium; N-alkyl-N-piperidino; N-alkyl-N-morpholino; and N-azabicyclo[2.2.2]octyl can optionally be substituted on carbon with one or more alkyl, hydroxyalkyl, hydroxy, COOR_{d}, or NR_{d}Rₑ. Rₐ, R_{b}, and R_{c} are each independently aryl or (C₁-C₂₄)alkyl; wherein Rd and Re are each independently hydrogen or alkyl. Any alkylene or alkyl can optionally be substituted on carbon with one or more oxo, hydroxy, halo, nitro, cyano, trifluoromethyl, COOR_{d}, or —NR_{d}Rₑ, and optionally interrupted with one or more oxy, imino, or thio, and where any alkyl or alkylene can optionally be partially unsaturated.

Another specific embodiment of the compound of formula (VI) is the compound wherein R₁, R₂, R₃, and R₄ are together —O—CH₂—.

Another specific embodiment of the compound of formula (VI) is the compound wherein R₅ is oxy.

Another specific embodiment of the compound of formual (VI) is the compound wherein R₆ is acetyl.

Another specific embodiment of the compound of formual (VI) is the compound wherein R₇ is N-diazabicyclo[2.2.2]octyl; N-pyridinium; or —N⁺(CH₃)₃.

In particular embodiments of the invention, the compound of formula (VI) is:
1-[(19β,28-epoxy-18α-oleanan-3β-yl)oxycarbonylmethyl]-4-aza-1-azonia-bicyclo[2.2.2]octane;
[(19β,28-epoxy-18α-oleanan-3β-yl)oxycarbonylmethyl]trimethylammonium; or
1-[(19β,28-epoxy-18α-oleanan-3β-yl)oxycarbonylmethyl]pyridinium.

A specific class of triterpene compounds present in the compositions of the instant invention include: Betulin; Lupeol; Lupeol acetate; Lupenone; 2-hydroxy-olean-1,2-ene-3-one-28,19-lactone; Allobetulinlactone; Allobetulonlactone; Allobetulinlactone trifluoroacetate; Allobetulinlactone phosphodichloride; 2-brom-Allobetulinlactone; Allobetulinlactone phosphate; Allobetulinlactone acetate; Allobetulin; Allobetulon; Allobetulin trifluoroacetate; Allobetulin phosphodichloride; Allobetulin phosphate; Allobetulin acetate; Allobetulon-1-ene-2-ol; 2-Br-Allobetulin; 3-TMS-O-Allobetulin; 3-aminomethyl-3hydroxy-Allobetulin; Allobetulon cyanohydrin; Allobetulin 3-tosylate; Betulon 28-acetate; Betulin 28-acetate; Betulonic aldehyde; Betulin dimesylate; Betulin-3-O-acetate-28-trifluoroacetate; Betulon; 3-O-acetyl-Betulinic aldehyde; Betulinic aldehyde; Betulon-1-ene-2-ol; Betulin ditrifluoroacetate; Betulin-28-tosylate; Betulin ditosylate; Betulinic acid; Betulonic acid; 3-O-acetyl-Betulinic acid; Betulin caffeate; Betulin dioxalyl chloride; Betulindiamine; Betulin 3-amine; Betulin 28-amine; Betulindihydroxyme; Betulindiphosphate; Betulindiphosphodichloride; Betulindiphosphate sodium salt; Betulin 3,28-bis((1R)-trans-chrysanthemate); Betulin 28-(1R)-trans-chrysanthemate; Betulin bis(N-pyridyl-2-acetate) dichloride; Betulin 3,28-diacrylate; Betulin 3,28-dimethacrylate; Betulin 28-acrylate-3-formiate; Betulin-28-monomethacrylate; Betulin-3,28-bis(P,P′-triphenylphosphinoacetate); Betuline-3,28-bis(tetramethylenediamino acetate); Betuline-3,28-bis(N,N′-diaza[2,2,2]bicyclooctanoacetate); Betulin-3,28-bis(N,N′-dibenzyldiazabicyclo[2.2.2]octanoacetate); Betulin-3,28-bis(N,N′-(4-oxybutyl)diazabicyclo[2.2.2]octanoacetate); Betulin-3,28-bis(oxyacetate); 3,28-Di(methylthiomethylene) betulin; 3-Methylthiomethyleneallobetulin; 28-Methylthiomethylenebetuline 3-acetate; 28-Methylthiomethylenebetul-3-one; Betulin 3-acetate-28-mesylate; Betulin 3,28-di(trifluoroacetamidglycinate); Betulin 28-trifluoroacetamidglycinate; Betulin 3,28-diacetylsalicilate; Betulin 3,28-di(2-oxyethylenoxyoxalate); Allobetulin 3-(poly(ethylene glycol)bis(carboxymethyl)ether)ester; Allobetulin 3-(poly(ethylene glycol)bis(carboxymethyl)ether)methyl ester; Betulin 3,28-di(poly(ethylene glycol)bis(carboxymethyl)ether)ester; Betulin 3,28-di(poly(ethylene glycol)bis(carboxymethyl)ether)ester; Betulin 3,28-di(poly(ethylene glycol)bis(carboxymethyl)ether)methyl ester; Poly(ethylene glycol)bis(carboxymethyl)ether 28,28′ dibetuline ester; Betulin 3,28-di(ethyl)carbamate; Betulin 3,28-disuccinate; Betulin 28-succinate; Betulin 3,28-disuccinyl dipoly(ethylene glycol)ester; 28,28′-Dibetulin poly(propylene glycol)toluene-2,4-dicarbamate terminated; Mixture of suberinic acids; cis-9,10-epoxy-18-hydroxyoctadecanoic acid; cis-9,10-epoxy-18-hydroxyoctadecanoic acid; cis-9,10-epoxy-18-hydroxyoctadecanoic acid+polyethyleneimine; cis-9,10-epoxy-18-hydroxyoctadecanoic acid+polyethyleneimine; cis-9,10-epoxy-18-hydroxyoctadecanoic acid+polyethyleneimine; 22-hydroxydocosanoic acid+polyethyleneimine; Dicarboxylic acids fraction+polyethyleneimine; Potassium salt of cis-9,10-epoxy-18-hydroxyoctadecanoic acid; 22-hydroxydocosanoic acid+polyethyleneimine; Docosandioic acid, 85%+polyethyleneimine; Lupeol 3-(polyethyleneimine propionate); cis-9,10-epoxy-18-hydroxyoctadecanoic acid+polyethyleneimine; Betulin 3,28-disuccinate+polyethyleneimine; Betulin 3,28-disuccinate+polyethyleneimine; Betulin 3,28-disuccinyl polyethyleneimine amide; Betulin 3,28-disuccinyl polyethyleneimine amide; Betulin 3,28-disuccinyl dichloride; Betulin 3,28-disuccinyl (1-methylpyrazine)amide; Lupeol 3-acrylate; cis-9,10-epoxy-18-acetoxyoctadecanoic acid; cis-9,10-epoxy-18-(m-nitrobezoiloxy)octadecanoic acid; cis-9,10-epoxy-18-acetoxyoctadecanoic acid (R)-(+)-α-phenylethylamide; cis-9,10-epoxy-18-(m-nitrobezoiloxy)octadecanoic acid (R)-(+)-α-phenylethylamide; cis-9,10-epoxy-18-hydroxyoctadecanoic acid (1)+polyethyleneimine; cis-9,10-epoxy-18-(3-acetoxylithocholioxy)octadecanoic acid methyl ester; Betulin 3,28-dimaleate+polyethylenimine; Betulin 3,28-dimaleate disodium salt; Betulin 3,28-dimaleate; 9,10,18-trihydroxyoctadecanoic acid; cis-9,10-epoxy-18-hydroxyoctadecanoic acid+polyethyleneimine; Betulin 3,28-diacetate; Betulin 3-acetate; Betulin 3,28-dibenzoate; Betulin 3-benzoate; Betulinic acid methyl ester; Betulin 3,28-di(2′-chloropropionate); Betulin 3,28-di(3′-chloropropionate); Betulin 3,28-di(4′-chlorobutyrate); bis(N,N′-pyridino-2-ethyl)betulin-3,28-carbamate dichloride; Betulin 3,28-di(4′-bromobutyrate); Betulin 3,28-di(2′-bromobutyrate); Betulin-3,28-bis(2-thiuroniumacetate)dihydrochloride; Betulin-3,28-bis (N,N′-pyridino-3-propionate) dichloride; Betulin-3,28-bis (N,N′-pyridino-2-propionate)dichloride; Betulin-3,28-bis (N,N′-pyridino-4-butyrate)dibromide; Betulin-3,28-bis (N,N′-pyridino-4-butyrate)dichloride; Betulin-3,28-bis (N,N′-pyridino-2-butyrate)dibromide; 1-(3,28-diacetoxylup-20-en-30-yl)-4-(dimethylamino)pyridinium bromide; Betulin-3-(N-DABCO-2-acetate); Betulin-3-chloroacetate; Betulin-3-(N-benzyl-N′-DABCO-2-acetate); Betulin-3-(N′-oxybutyl-N-DABCO-2-acetate); Mixture of betulin-3-phosphonoacetate and betulin-28-phosphonoacetate; Dihydro-29-carboxy-betulin; Dimethylamide dihydro-29-carboxybetulin; Betulin 3,28-disuccinyl di(4-methyl-4-benzylpyrazonium bromide) amide; 9,10,18-treo-trihydroxyoctadecanoic acid (Phloionolic acid); 22-Hydroxydocosanoic acid (IK32); Birch bark tannin; Birch bark tannin —Na salt; Birch bark tannin —K salt; Betulin-3,28-bis(benzyltetramethyl-ethylenediamino acetate chloride); Betuline-3,28-dioxalate; Betulin-28-maleate; Betulin-3,28-bis(diacetyltartrate); Betulin-3,28-bis(diacetyltartrate)disodium salt; N-(3,28-diacetoxylup-20-en-30-yl)-1,4-diazabicyclo[2.2.2]octane bromide; 3,28,30-triacetoxylup-20(29)-ene; 3,28-bis(3-(1-piperidinyl)propanoyloxy)lup-20(29)-ene dihydrochloride; 30-Bromo-3,28-dihydroxylup-20(29)-ene; 1-(3,28-dihydroxylup-20(29)-en-30-yl)-4-(dimethylamino)pyridinium bromide; 1-(lup-20(29)-en-30-yl)-1,4-diazabicyclo[2.2.2]octane bromide; S-(3,28-dihydroxylup-20(29)-en-30-yl)thiuronium bromide; 1-(3,28-dihydroxylup-20(29)-en-30-yl)-pyridinium bromide; 1-(3,28-dihydroxylup-20(29)-en-30-yl)-3,5-dimethylpyridinium bromide; Adduct of 1 mole of betulin-3-chloroacetate and 1 mole of SV-23; betulin-3,28-bis(2-thiuroniumacetate)dihydrochloride; lup-20(29)-ene-3,28-bis(N,N′-4-dimethylaminopyridino-2-propionate) dichloride; lup-20(29)-ene-3,28-bis(N,N′-1,4-diazabicyclo[2.2.2]octane-2-propionate)dichloride; lup-20(29)-ene-3,28-bis(thiuronium-4-butirate)dichloride; 1-(3,28-dihydroxylup-20(29)-en-30-yl)-4-(hydroxymethyl)pyridinium bromide; 1-(3,28-dihydroxylup-20(29)-en-30-yl)-3-hydroxy-1-azabicyclo[2.2.2]octane bromide; 3,28-dihydroxy-30-(1,2,4-triazol-1-yl)-lup-20(29)-ene; 22-hydroxydocosanoic acid sodium salt; 22-hydroxydocosanoic acid potassium salt; 9,10,18-trihydroxyoctadecanoic acid sodium salt; 9,10,18-trihydroxyoctadecanoic acid potassium salt; 9,10-epoxy-18-hydroxyoctadecanoic acid sodium salt; 9,10-epoxy-18-hydroxyoctadecanoic acid potassium salt; lup-20(29)-ene-3,28-bis(N,N′-1,4-diazabicyclo[2.2.2]octane-4-butyrate) dibromide; lup-20(29)-ene-3,28-bis(N,N′-1,4-diazabicyclo[2.2.2]octane-4-butyrate)dichloride; Bis(N,N′-1,4-diazabicyclo[2.2.2]octane-2-ethyl)-lup-20(29)-ene-3,28-carbamate dichloride; 30-Bromo-3,28-bis(chloroacetyl)lup-20(29)-ene; 1-(3,28-diacetoxylup-20(29)-en-30-yl)-pyridinium bromide; 1-(3,28-dihydroxylup-20(29)-en-30-yl)-3-(hydroxymethyl)pyridinium bromide; lup-20(29)-en-3,28-bis(pyridylmethylurea acetate)dichloride; lup-20(29)-en-3,28-bis(3-oxymethylpyredyniumacetoxy)dichloride; lup-20(29)-en-3,28-bis(2-oxymethylpyredyniumacetoxy) dichloride; lup20(29)-ene-3,28-bis (N,N′-4-dimethylaminopyridino-3-propionate)dichloride; lup20(29)-ene-3,28-bis (N,N′-4-dimethylaminopyridino-4-butyrate)dibromide; lup20(29)-ene-3,28-bis(N,N′-4-dimethylaminopyridino-2-butyrate)dibromide; lup-20(29)-ene-3,28-bis(N,N′-1,4-diazabicyclo[2.2.2]octane-2-butyrate)dibromide; betulin 3-mono(N-pyridyacetate)chloride; lup-20(29)-en-3 mono (2-oxymethylpyredyniumacetoxy)chloride; Betulin 3, 28-bis(chloroacetate)dichloride+4-Hydroxy-1-methylpiperidine; Betulin 3,28 bis (chloroacetate) dichloride+4-methylmorpholine; lup-20(29)-en-3 mono(pyridylmethylurea acetate)chloride; 3,28,30-Trihydroxylup-20(29)-ene; Lup20(29)-ene-3,28-bis (2,4-lutidine-1-acetate)dichloride; lup20(29)-ene-3,28-bis (3,5-lutidine-1-acetate)dichloride; lup20(29)-ene-3,28-bis (4-(dimethylamino)-1-(acetate)pyridine) dichloride; lup20(29)-ene-3-(2-Picoline-1-acetate) chloride; lup20(29)-ene-3-mono (2,4-lutidine-1-acetate) chloride; lup20(29)-ene-3 (4-hydroxy-1-Methyl,1-acetate piperidine)chloride; lup20(29)-ene-3 (4′-Methylmorpholine-1′-acetate)chloride; lup20(29)-ene-3 (3,5-lutidine-1-acetate)chloride; lup20(29)-ene-3(4-(dimethylamino)-1-(acetate)pyridine)chloride; Betulin 3,28 bis(octhyldimethylamoniumacetoxy)dichloride; Betulin 3(octhyldimethylamoniumacetoxy)chloride; Betulin 3,28-bis(tetradecyldimethylamoniumacetoxy)dichloride; Betulin 3 (tetradecyldimethylamoniumacetoxy)chloride; 3,28-dihydroxy-30-(imidazol-1-yl)-lup-20(29)-ene; 3,28-diacetoxy-30-(triazol-1-yl)-lup-20(29)-ene; Betulin-3-(2-chloropropionate); Betulin-3-(N-1-triazolylacetate); Betulin-3-(N−1-triazolyl)-2-propionate; Betulin-3,28-bis(bromoacetate); 3-Acetoxylup-20(29)-ene-28-aldoxyme; 3-Acetoxylup-20(29)-ene-28-aldmethoxyme; Lup-20(29)-ene-3-one-28-al dioxyme; Lup-20(29)-ene-3-one-28-al dimethoxyme; 3-(1,2,4-Triazol-1-yl)acetylallobetulin; 3-(2-(1,2,4-Triazol-1-yl)propionyl)allobetulin; Lup-20(29)-ene-3-acetate-28-p-nitrobenzoate; Lup-20(29)-ene-3-acetate-28-o-nitrobenzoate; Lup-20(29)-ene-3-acetate-28-m-nitrobenzoate; Betulin-3-(N-1-pyrazolyl)-2-propionate; 3,28-bis(2-(triazol-1-yl)propionate)betulin; 28-(2-Chloropropionyl)betulin; 28-(2-(triazol-1-yl)propionyl)betulin; 3,28-bis(2-(imidazol-1-yl)propionyl)betulin; 3,28-Dimethylbetulin; 3-((Imidazol-1-yl)acetoxy)-19β,28-epoxy-18α-oleanan; 3-[2-(Imidazol-1-yl)propionyloxy]-19β,28-epoxy-18α-oleanan; 3-((Pyrazol-1-yl)acetoxy)-19β,28-epoxy-18α-oleanan; 3-[2-(Pyrazol-1-yl)propionyloxy]-19β,28-epoxy-18α-oleanan; 28-(2-imidazolylpropionyloxy)lup-20 (29)-ene; 1-(3,28-dihydroxylup-20(29)-en-30-yl)piperidine; 1-(3,28-diacetoxylup-20(29)-en-30-yl)piperidine; 3,28,30-tris(chloroacetoxy)lup-20(29)-ene; 3β-(N-diazabicyclo[2.2.2]octylacetyloxy)-19β,28-epoxy-18α-oleanan bromide; 3β-(N-diazabicyclo[2.2.2]octylacetyloxy)-19β,28-epoxy-18α-oleanan chloride; 3β-(N-pyridiniumacetyloxy)-19β,28-epoxy-18α-oleanan bromide; 3β-(N-pyridiniumacetyloxy)-19β,28-epoxy-18α-oleanan chloride; 3β-[-(N′,N′-dimethylaminopyridinium)-N-acetyloxy]-19β,28-epoxy-18α-oleanan bromide; 3β-[-(N′,N′-dimethylaminopyridinium)-N-acetyloxy]-19β,28-epoxy-18α-oleanan chloride; 3β-(N-octyldimethylaminoacetyloxy)-19β,28-epoxy-18α-oleanan bromide; 3β-[N-(2-hydroxyethyl)laminoacetyloxy]-19β,28-epoxy-18α-oleanan bromide; 3β-[N,N-dimethyl-N-(2-hydroxyethyl)aminoacetyloxy]-19β,28-epoxy-18α-oleanan bromide; 3β-[N,N-dimethyl-N-(2-hydroxyethyl)aminoacetyloxy]-19β,28-epoxy-18α-oleanan chloride; 3β-[N-(3-hydroxymethylpyridinium)acetyloxy]-19β,28-epoxy-18α-oleanan bromide; 3β-[(N,N,N′,N-′tetramethylethylenediamino)acetyloxy]-19β,28-epoxy-18α-oleanan bromide; 3,28-dimethoxy-30-bromobetulin; combinations thereof; and pharmaceutically acceptable salts thereof.

The compounds present in the compositions of the instant invention can comprise one triterpene moiety derivatized with one or more quaternary ammonium group (e.g., N⁺-containing group). Preferred N⁺-containing groups include N⁺-containing heteraryl, N⁺-containing heterocycle, or —NRₐR_{b}R_{c}, wherein Rₐ, R_{b}, and R_{c} are each independently (C₁-C₂₄)alkyl, aryl, arylalkyl, heteroarylalkyl, heterocycle, or hetercyclealkyl. Preferably, a single triterpene moiety is derivatized with one, two, three, or four N⁺-containing groups.

The compounds present in the compositions of the instant invention can also comprise more than one triterpene moiety derivatized to a single N⁺-containing group and comprise oligomers of alternating triterpene moieties and N⁺-containing groups. In these cases, the triterpene moieties can be further derivatized with additional N⁺-containing groups.

For instance, one embodiment of the invention provides a composition that includes a compound of formula (VII) or (VIII): 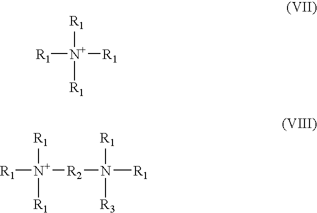
Each R₁ is independently (C₁-C₂₄)alkyl or is alkylcarbonyl attached through the carbonyl to the oxy at the 3 or 28 carbon of betutlin, lupeol, or allobetulin, or to an imino or thio in place of the oxy at the 3 or 28 carbon of betulin, lupeol, or allobetulin, wherein if it is attached to an oxy, imino, or thio at the 28 carbon of allobetulin, carbon 19 is a methylene. R₂ is (C₁-C₂₄)alkyl. R₃ is absent or (C₁-C₂₄)alkyl or is alkylcarbonyl attached through the carbonyl to the oxy at the 3 or 28 carbon of betulin, lupeol, or allobetulin, or to an imino or thio in place of the oxy at the 3 or 28 carbon of betulin, lupeol, or allobetulin, wherein if it is attached to an oxy, imino, or thio at the 28 carbon of allobetulin, carbon 19 is a methylene. Any alkyl or alkylcarbonyl can optionally be substituted with one or more oxo, hydroxy, mercapto, or NR_{d}Rₑ. R_{d} and Rₑ are each independently hydrogen or alkyl. The compound in this case comprises at least two moieties selected from the group of betulin, allobetulin, and lupeol.

In one specific embodiment of the compound of formula (VIII), the compound is N,N,N′,N′-tetramethylethylenediamine-N,N′-bis-[lup-20(29)-ene-3-acetate].

In one embodiment, the compounds present in the compositions of the instant invention include one or more triterpene moieties covalently attached via a linker to a quaternary ammonium salt. The linker can attach to the triterpene moiety at any suitable position of the triterpene. The linker can attach to the quaternary ammonium salt at the N⁺ atom or at any other suitable position. The linker can be, for instance, alkylene, alkylcarbonyl, alkoxy, alkylimino, oxyalkylcarbonyl, carbonylalkylcarbonyl, or carbonylalkyloxy.

The quaternary ammonium salt can also be attached directly to the triterpene without a linker. The attachment in this case can be at any suitable position of the triterpene and any suitable position of the quaternary ammonium salt.

A specific method of the invention is the method of treating a mammal afflicted with a fungal infection comprising administering to the mammal a composition that includes an essential oil and an effective anti-fungal amount of a compound of formula (I)-(VI), wherein the mammal is a human.

Another specific method of the invention is the method of treating a mammal afflicted with a fungal infection comprising administering to the mammal a composition that includes an essential oil and an effective anti-fungal amount of a compound of formula (I)-(VI), wherein the fungal infection is caused by a dermatophytic fungus.

Another specific method of the invention is the method of treating a mammal afflicted with a fungal infection comprising administering to the mammal a composition that includes an essential oil and an effective anti-fungal amount of a compound of formula (I)-(VI), wherein the fungal infection is caused by a dermatophytic fungus that is Microsporum canis, Microsporum gyseum, Microsporum audouinii, Trichophyton tonsurans, Trichophyton mentagrophytes, Epidermophyton floccosum, Trichophyton rubrum, or Pityrosporum ovale.

Another specific method of the invention is the method of treating a mammal afflicted with a fungal infection comprising administering to the mammal a composition that includes an essential oil and an effective anti-fungal amount of a compound of formula (I)-(VI), wherein the fungal infection is caused by Candida albicans or Candida guilliermoundi.

Another specific method of the invention is the method of treating a mammal afflicted with a fungal infection comprising administering to the mammal a composition that includes an essential oil and an effective anti-fungal amount of a compound of formula (I)-(VI), wherein the fungal infection is caused by Blastomyces dermatidis or Cryptococcus neoformans.

Another specific method of the invention is the method of inhibiting or killing a fungus comprising contacting the fungus or yeast with a composition that includes an essential oil and an effective anti-fungal amount of a compound of formula (I)-(VI), wherein the fungus is a dermatophytic fungus.

Another specific method of the invention is the method of inhibiting or killing a fungus comprising contacting the fungus with an effective anti-fungal amount of a composition that includes an essential oil and an effective anti-fungal amount of a compound of formula (I)-(VI), wherein the fungus is a dermatophytic fungus that is Microsporum canis, Microsporum gyseum, Microsporum audouinii, Trichophyton tonsurans, Trichophyton mentagrophytes, Epidermophyton floccosum, Trichophyton rubrum, or Pityrosporum ovale.

Another specific method of the invention is the method of inhibiting or killing a fungus comprising contacting the fungus with an effective anti-fungal amount of a composition that includes an essential oil and an effective anti-fungal amount of a compound of formula (I)-(VI), wherein the fungus is Candida albicans or Candida guilliermoundi.

Another specific method of the invention is the method of inhibiting or killing a fungus comprising contacting the fungus with an effective anti-fungal amount of a composition that includes an essential oil and an effective anti-fungal amount of a compound of formula (I)-(VI), wherein the fungus is Blastomyces dermatidis or Cryptococcus neoformans.

Processes for preparing the triterpenes employed in the invention (i.e., compounds of formula (I)-(VI)) are provided as further embodiments of the invention and are illustrated by the following procedures in which the meanings of the generic radicals are as given above unless otherwise qualified. Specifically, the compounds of formula (I)-(VI) can be prepared from convenient starting materials, employing procedures (e.g., reagents and reaction conditions) known to those of skill in the art. For example, suitable reagents and reaction conditions are disclosed, e.g., in Advanced Organic Chemistry, Part B: Reactions and Synthesis, Second Edition, Carey and Sundberg (1983); Advanced Organic Chemistry, Reactions, Mechanisms, and Structure, Second Edition, March (1977); Greene, T. W., Protecting Groups In Organic Synthesis, Third Edition, 1999, New York, John Wiley & sons, Inc.; and Comprehensive Organic Transformations, Second Edition, Larock (1999).

In cases where compounds are sufficiently basic or acidic to form stable nontoxic acid or base salts, administration of the compounds as salts may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compositions that include an essential oil and a compound of formula (I)-(VI) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compositions can be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compositions may be combined with one or more excipients and used in the form of ingestible tablets, buccal tablets, troches, capsules, elixirs, suspensions, syrups, wafers, and the like. Such preparations should contain at least 0.1% of the triterpene compound. The percentage of the compositions can, of course, be varied and may conveniently be between about 2 to about 60% of the weight of a given unit dosage form. The amount of active compound (i.e., triterpene compound) in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound (i.e., triterpene), sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound (i.e., triterpene) may be incorporated into sustained-release preparations and devices.

The composition may also be administered intravenously or intraperitoneally by infusion or injection. Solutions of the triterpene and essential oil can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the triterpene and essential oil in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the triterpene and essential oil, plus any additional desired ingredient present in the previously sterile-filtered solutions.

For topical administration, the present compositions may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the triterpene and essential oil can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compositions of the triterpene and essential oil, to the skin, are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compositions of the triterpene and essential oil can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compositions of the triterpene and essential oil in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the triterpene, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The composition is conveniently administered in unit dosage form; for example, containing 5 to 1000 mg, conveniently 10 to 750 mg, most conveniently, 50 to 500 mg of triterpene per unit dosage form.

Ideally, the composition should be administered to achieve peak plasma concentrations of the triterpene of from about 0.5 to about 75 μM, preferably, about 1 to 50 μM, most preferably, about 2 to about 30 μM. This may be achieved, for example, by the intravenous injection of a 0.05 to 5% solution of the triterpene, optionally in saline, or orally administered as a bolus containing about 1-100 mg of the triterpene. Desirable blood levels may be maintained by continuous infusion to provide about 0.01-5.0 mg/kg/hr or by intermittent infusions containing about 0.4-15 mg/kg of the triterpene(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a composition of the invention to act as an anti-fungal agent may be determined using pharmacological models which are well known to the art.

The compositions of the invention may be also be useful as pharmacological tools for the further investigation of the mechanism of their anti-fungal action.

The compositions of the invention can also be administered in combination with other therapeutic agents that are effective to treat fungal infections, or to inhibit or kill a fungus.

The system used to name the triterpenes employed in the compositions of the invention will be clear to one of skill in the art based on the following examples. Names generally consist of the base structure, e.g., betulin, allobetulin, or lupeol, followed by a substituent. For example, betulin-28-succinate consists of a succinic acid molecule esterified to the hydroxyl at carbon 28 of betulin. If no number is given for the substituent, the substituent is attached to the hydroxyl at carbon 3 on the base structure.

Betulin-3-glycerol oxalate is a compound of formula (I), wherein R₄ and R₅ together are hydrooxyl, R₂ and R₃ together are —OC(═O)C(═O)OCH₂CH(OH)CH₂OH, and R₁ is hydrogen. Betulin-1-ene-2-ol is a compound of formula (I), wherein the bond between carbons 1 and 2 is a double bond, R₁ is hydroxyl, R₂ and R₃ together are hydroxymethyl, and R₄ and R₅ together are oxo. Uvaol is a compound of formula (II), wherein R₁₀ is methyl, R₉ is hydrogen, R₈ is methyl, R₇ is hydrogen, R₁₁ is hydroxymethyl, R₆ is absent and the bond between carbons 12 and 13 is double, R₃ is hydrogen, R₄ and R₅ are methyl, R₂ is hydrogen, and R₁ is hydroxy. Oleanolic acid has the same structure as uvaol, except it has a carboxy at R₁, instead of hydroxymethyl. The structure of hederin hydrate is disclosed at page 871 of the Aldrich Chemical Co. 2000-2001 catalog. The structure of other named compounds can be found in standard sources such as the Merck Index. “Betulin arabinose galactan” refers to betulin in a solution of arabino-galactan.

Unless otherwise stated, amino acid substituents are attached to the compounds of the invention through their carboxyl groups via ester linkages. Thus, betulin-3,28-diglycine is the same compound as betulin-3,28-diglycine ester.

The compositions of the present invention can further optionally include an anti-infective agent. Suitable anti-infective agents include, for example:
[1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-Amino-3,6-dideoxy-β-D-mannopyranosyl)oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid (Amphotericin B);
5-fluorocytosine (Flucytosine);
2,4-difluoro-α,α³-bis(1H-1,2,4-triazol-1-ylmethyl) benzyl alcohol) (Fluconazole);
griseofulvin microsize (Griseofulvin);
(E)-N-(6,6-dimethyl-2-hepten-4-ynyl)-N-methyl-1-naphthalenemethanamine hydrochloride)(Terbinafine);
cis-1-acetyl-4-[4-[(2-(2,4-dichlorophenyl)-2-(1H-imadazol-1-ylmethyl)-1,3-dioxolan-4-yl]methoxyl]phenyl]piperazine (Ketoconazole);
(±)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methyoxy]phenyl]-1-piperazinyl]phenyl]-Δ²-1,2,4-triazolin-5-one mixture with (±)-1-[(R*)-sec-butyl]-4-[p-[4-[p-[[(2S*, 4R*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one or (±)-1-[(RS)-sec-butyl]-4-[p-[4-[p-[[(2R, 4S)-2-(2,4-dichlorophenyl)-2-(1H-1,2,4-triazol-1-ylmethyl)-1,3-dioxolan-4-yl]-methoxy]phenyl]-1-piperazinyl]phenyl]-Δ²-1,2,4-triazolin-5-one (Itraconazole);
2-chloro-5-hydroxy-1,3-dimethylbenzene (Chloroxylenol);
griseofulvin ultramicrosize (Griseofulvin);
(E)-N-(6,6,-dimethyl-2-hepten-4-ynyl)-N-methyl-1-naphthalenemanamine hydrochloride (Terbinafine);
6-cyclohexyl-1-hydroxy-4-methyl-2(1H)-pyridinone (Ciclopirox);
N-4-tert-butyl-benzyl-N-methyl-1-naphthalenemethylamine hydrochloride (Butenafine hydrochloride);
nystatin;
(E)-N— (Cinnamyl-N-methyl-1-naphthalenemethylamine hydrochloride (Naftifine hydrochloride);
2′,4′-dichloro-2-imidazol-1-ylacetophenone (Z)-[0-(2,4-dichlorobenzyl)oxime]mononitrate (Oxiconazole nitrate),
6-cyclohexyl-1-hydroxy-4-methyl-2(1H)-pyridone (Ciclopirox);
selenium sulfide;
(±)-1-[4-(p-chlorophenyl)-2-[(2,6-dichlorophenyl)thio]butyl]imidazole mononitrate (Butoconazole nitrate);
([1-(o-chloro-.,.-diphenylbenzyl)imidazole]) (Clotrimazole);
(cis-1-[p-[[2-(2,4-dichlorophenyl)-2-(1H-1,2,4-triazol-1-ylmethyl)-1,3-dioxolan-4-yl]methoxy phenyl]-4-isopropyl-piperazine (Tercanazole);
6-cyclohexyl-1-hydroxy-4-methyl-2(1H)-pyridone (ciclopirox);
and combinations thereof.

All patents, patent docuements, and references cited herein are incporporated by reference.

1. A pharmaceutical composition comprising a triterpene and an essential oil.

2. The composition of claim 1 wherein the triterpene is a compound of formula (I): 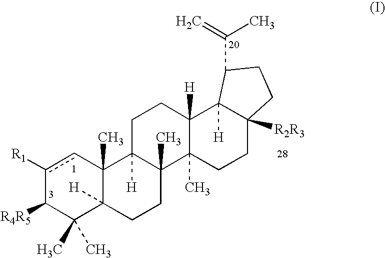
wherein
R₁ is hydrogen or hydroxy;
R₂ is a direct bond, carbonyl, oxy, thio, carbonyl oxy, oxy carbonyl, (C₆-C₁₀)aryl, or (C₁-C₆)alkyl;
R₃ is hydrogen, hydroxy, hydroxy(C₁-C₆)alkyl, (C₁-C₆)alkyl, O═P(OH)₂, O═P(OH)₂OP(O)(OH)—, (C₁-C₅)alkanoyl, Si(R)₃ wherein each R is H, phenyl or (C₁-C₆)alkyl, C(O)N(R)₂, benzyl, benzoyl, tetrahydropyran-2-yl, 1-[(C₁-C₄)alkoxy](C₁-C₄)alkyl, or a glycoside;
R₄ is hydrogen, hydroxy, hydroxy(C₁-C₆)alkyl, (C₁-C₆)alkyl, O═P(OH)₂, O═P(OH)₂OP(O)(OH)—, (C₁-C₅)alkanoyl, Si(R)₃ wherein each R is H, phenyl or (C₁-C₆)alkyl, C(O)N(R)₂, benzyl, benzoyl, tetrahydropyran-2-yl, 1-[(C₁-C₄)alkoxy](C₁-C₄)alkyl, or a glycoside; or R₄ and R₅ together are oxo; and
R₅ is direct bond, carbonyl, oxy, thio, carbonyl oxy, oxy carbonyl, (C₆-C₁₀)aryl, or (C₁-C₆)alkyl; or R₄ and R₅ together are oxo;
wherein any alkyl can optionally be substituted with one or more halo, hydroxy, (C₆-C₁₀)aryl, nitro, cyano, (C₁-C₆)alkoxy, trifluoromethyl, polyethyleneimine, poly(ethylene glycol), oxo, NR₇R₈, wherein R₇ and R₈ are each independently hydrogen, (C₁-C₆)alkyl or polyethyleneimine; or C(═O)OR₉, wherein R₉ is hydrogen, (C₁-C₆)alkyl, or polyethyleneimine;
each of the bonds represented by - - - is independently absent or is present;
wherein any alkyl is optionally interrupted on carbon with one or more oxy, thio, sulfinyl, sulfonyl, polyethyleneimine, or poly(ethylene glycol);
wherein any alkyl is optionally partially unsaturated;
wherein any aryl can optionally be substituted with one or more halo, hydroxy, nitro, cyano, (C₁-C₆)alkoxy, trifluoromethyl, polyethyleneimine, poly(ethylene glycol), oxo, NR₇R₈, wherein R₇ and R₈ are each independently hydrogen, (C₁-C₆)alkyl or polyethyleneimine; or C(═O)OR₉, wherein R₉ is hydrogen, (C₁-C₆)alkyl, or polyethyleneimine;
or a pharmaceutically acceptable salt thereof.

3. The composition of claim 2 wherein the bond between carbons 1 and 2 is a single bond.

4. The composition of claim 2 wherein the bond between carbons 1 and 2 is a double bond.

5. The composition of claim 2 wherein R₁ is hydrogen.

6. The composition of claim 2 wherein R₁ is hydroxy.

7. The composition of claim 2 wherein R₂ is a direct bond.

8. The composition of claim 2 wherein R₃ is (C₁-C₆)alkyl; wherein
any alkyl can optionally be substituted with one or more oxo, carboxy, amino, —OP(═O)(OH)₂, or phenyl;
any alkyl is optionally interrupted on carbon with one or more oxy or thio;
any alkyl is optionally partially unsaturated; and
any aryl can optionally be substituted with one or more hydroxy or carboxy.

9. The composition of claim 8 wherein R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂C(═O)C(═O)—(—NHCH₂CH₂)ₓ—[—N(CH₂CH₂NH₂)CH₂CH₂]_{y}.

10. The composition of claim 2 wherein R₄ is hydrogen or (C₁-C₆)alkyl; wherein
any alkyl can optionally be substituted with one or more oxo, carboxy, amino,
—OP(═O)(OH)₂, or phenyl;
any alkyl is optionally interrupted on carbon with one or more oxy or thio;
any alkyl is optionally partially unsaturated; and
any aryl can optionally be substituted with one or more hydroxy or carboxy.

11. The composition of claim 10 wherein R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)ₓ—[—N(CH₂CH₂NH₂)CH₂CH₂]_{y}.

12. The composition of claim 2 wherein R₅ is oxy.

13. The composition of claim 2 wherein R₄ and R₅ together are oxo.

14. The composition of claim 2 wherein
R₁ is hydrogen or hydroxy;
R₂ is a direct bond;
R₃ is (C₁-C₆)alkyl;
R₄ is hydrogen or (C₁-C₆)alkyl; and
R₅ is oxy or R₄ and R₅ together are oxo;
wherein
any alkyl can optionally be substituted with one or more oxo, carboxy, amino,
—OP(═O)(OH)₂, or phenyl;
any alkyl is optionally interrupted on carbon with one or more oxy or thio;
any alkyl is optionally partially unsaturated; and
any aryl can optionally be substituted with one or more hydroxy or carboxy.

15. The composition of claim 2 wherein
R₁ is hydrogen or hydroxy;
R₂ is a direct bond;
R₃ is hydroxymethyl, (carboxymethoxy)acetoxymethyl, 4-carboxybutanoyloxymethyl, 3-carboxypropenoyloxymethyl, 2-carboxybenzoyloxymethyl, 3-carboxypropanoyloxymethyl, aminoacetoxymethyl, carboxycarbonyloxymethyl, 2-amino-3-methyl-butanoyloxymethyl, 4-carboxy-(3,3-dimethyl)butanoyloxymethyl, or —CH₂C(═O)C(═O)—(—NHCH₂CH₂)ₓ—[—N(CH₂CH₂NH₂)CH₂CH₂]_{y};
R₄ is hydrogen, hydroxymethyl, (carboxymethoxy)acetyl, 4-carboxybutanoyl, 3-carboxypropenoyl, 2-carboxybenzoyl, 3-carboxypropanoyl, aminoacetyl, carboxycarbonyl, 2-amino-3-methyl-butanoyl, 4-carboxy-(3,3-dimethyl)butanoyl, 3-carboxy-3-methylbutanoyl or —C(═O)C(═O)—(—NHCH₂CH₂)ₓ—[—N(CH₂CH₂NH₂)CH₂CH₂]_{y}; and
R₅ is oxy or R₄ and R₅ together are oxo.

16. The composition of claim 2 wherein the triterpene is betulin; betulin-3,28-diglycine; betulin-28-glycerol oxalate; betulin-28-glycine; betulin-28-oxalate; betulin arabinose galactan; betulin-3,28-diglycolate; betulin-3-maleate; betulin-3,28-di-(L-glutamic acid γ-benzylester) ester; betulin-3,28-di-L-alanine; betulin-3,28-di-L-proline ester; betulin-3,28-dioxalate; betulin-1-ene-2-ol; betulin-3,28-diphenylalanine; betulin-3,28-dioxalate-polyethylene amine; betulin-3,28-diphosphate; betulin-3-caffeate; betulin-3,28-(3′,3′-dimethyl)glutarate; betulin-28-diglycolate; betulin-28-glutarate; betulin-28-maleate; betulin-28-phthalate; betulin-3,28-di(3′,3′-dimethyl)glutarate; betulin-3,28-didiglycolate; betulin-3,28-dithiodiglycolate; betulin-3,28-diglutarate; betulin-3,28-dimaleate; betulin-3,28-diglycolate; betulin-3,28-diphthalate; betulin-3,28-di-L-valine ester; betulin-28-succinate; betulin-3,28-disuccinate; betulin-3,28-di-(polyethylene glycol)-COOH (Mw=1448); betulin-3,28-di-(polyethylene glycol)-COOH (Mw=906); betulin-3,28-di-(polyethylene glycol)-COOH (Mw=906); betulinic acid; betulon-1-ene-2-ol; betulin-3,28-(dipoly(ethylene glycol)bis (carboxymethylester); hederin hydrate; lupeol; lupeol-3-glutarate; lupeol-3-succinate; lupeol-3-thiodiglycolate; lupeol-3-phthalate; oleanolic acid; ursolic acid; uvaol; betulin oxalate; betulin di-(L-glutamic acid γ-benzylester)ester; betulin3,28-di-L-proline; betulin-3,28-diphenylalanine ester; betulin-3,28-phosphate; betulin-3,28-dioxalate-3-polyethyleneimine; betulin-3,28-di(31,31-dimethyl)glutarate; betulin-3,28-dioxalate-3,28-polyethyleneimine; betulin-3,28-di-L-valine; lupeol-3-amine; lupeol-3-(3′,3′-dimethyl)succinate; lupeol-3-maleate; lupenone; or lupenon-1,2-ene-2-ol.

17. The composition of claim 1 wherein the triterpene is a compound of formula (II): 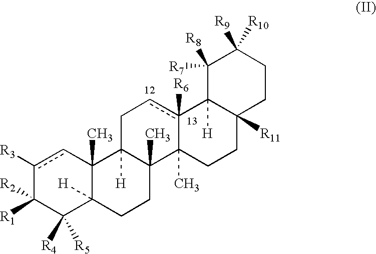
wherein
one of R₁ and R₂ is —O—Y and the other is hydrogen or (C₁-C₆)alkyl optionally substituted by hydroxy, (C₁-C₆)alkoxy, halo, halo(C₁-C₆)alkoxy or NRⱼRₖ wherein Rⱼ and Rₖ are independently H, (C₁-C₆)alkyl or (C₁-C₆)alkonyl; or R₁ and R₂ together are oxo (═O);
R₃ is hydrogen, halo, carboxy, mercapto, (C₁-C₆)alkyl, (C₃-C₈)cycloalkyl, or —O—Y;
R₄ and R₅ are each independently hydrogen, (C₁-C₆)alkyl, or hydroxy(C₁-C₆)alkyl;
R₆ is hydrogen or is absent when the adjacent - - - is a bond;
R₇ is hydrogen or (C₁-C₆)alkyl;
R₈ is hydrogen, (C₁-C₆)alkyl, or hydroxy(C₁-C₆)alkyl and R₁₁ is hydrogen, (C₁-C₆)alkyl, carboxy, or hydroxy(C₁-C₆)alkyl; or R₈ and R₁₁ together are —O—C(═X)—;
R₉ and R₁₀, are each independently hydrogen or (C₁-C₆)alkyl;
each of the bonds represented by - - - is independently absent or is present;
X is two hydrogens, oxo (═O) or thioxo (═S);
each Y is independently H, aryl, P(O)(Cl)₂, (C₃-C₈)cycloalkyl, adamantyl, —SO₂Rₐ O═P(R_{b})₂, O═P(R_{c})₂OP(O)(R_{d})—, Si(Rₑ)₃, tetrahydropyran-2-yl, an amino acid, a peptide, a glycoside, or a 1 to 10 membered branched or unbranched carbon chain optionally comprising 1, 2, or 3 heteroatoms selected from non-peroxide oxy, thio, and —N (R_{f})—; wherein said chain may optionally be substituted on carbon with 1, 2, 3, or 4 oxo (═O), hydroxy, carboxy, halo, mercapto, nitro, —N(R_{g})(Rₕ), (C₃-C₈)cycloalkyl, (C₃-C₈)cycloalkyloxy, aryl, aryloxy, adamantyl, adamantyloxy, hydroxyamino, trifluoroacetylamino, a glycoside, an amino acid, or a peptide; and wherein said chain may optionally be saturated or unsaturated (e.g. containing one, two, three or more, double or triple bonds);
Rₐ is (C₁-C₆)alkyl or aryl;
R_{b}, R_{c}, and R_{d} are each independently hydroxy, (C₁-C₆)alkoxy, hydroxy(C₂-C₆)alkoxy, adamantyloxy, adamantyl (C₁-C₆)alkoxy, norbornyloxy, 1,1-di(hydroxymethyl)-2-hydroxyethoxy, carboxy(C₁-C₆)alkoxy, 2,3-epoxypropyloxy, benzyloxy, (C₃-C₈)cycloalkyloxy, NRₓR_{y}, or aryloxy;
Rₑ is H, aryl or (C₁-C₆)alkyl;
R_{f} is hydrogen, (C₁-C₆)alkyl, (C₁-C₆)alkanoyl, phenyl or benzyl;
R_{g} and Rₕ are each independently selected from the group consisting of hydrogen, (C₁-C₆)alkyl, hydroxy(C₁-C₆)alkyl, adamantyl, adamantyl(C₁-C₆)alkyl, amino(C₁-C₆)alkyl, aminosulfonyl, (C₁-C₆)alkanoyl, aryl and benzyl; or R_{b} and R_{c} together with the nitrogen to which they are attached form a pyrrolidino, piperidino, or morpholino radical; and
Rₓ and R_{y} are each independently hydrogen, (C₁-C₆)alkyl, (C₁-C₆)alkanoyl, aryl or benzyl;
wherein each aryl of Y, Rₐ-R_{d}, R_{g}-Rₕ, Rₓ, and R_{y} may optionally be substituted by 1, 2, or 3 aminosulfonyl, carboxy, NRᵢRⱼ, (C₁-C₆)alkyl, (C₁-C₆)alkoxy, hydroxy, halo, nitro, cyano, mercapto, carboxy, hydroxy(C₁-C₆)alkyl, halo(C₁-C₆)alkyl, trifluoromethoxy, (C₁-C₆)alkanoyl, (C₁-C₆)alkoxycarbonyl, (C₁-C₆)alkylthio, or (C₁-C₆)alkanoyloxy; wherein Rᵢ and Rⱼ are each independently hydrogen, (C₁-C₆)alkyl, (C₁-C₆)alkanoyl, phenyl, or benzyl;
wherein any alkyl can optionally be substituted with one or more polyethyleneimine or poly(ethylene glycol); and wherein any alkyl can optionally be interrupted with one or more polyethyleneimine or poly(ethylene glycol);
or a pharmaceutically acceptable salt thereof.

18. The composition of claim 17 wherein the bond between carbons 1 and 2 is a single bond.

19. The composition of claim 17 wherein R₁ is —O—Y and Y is hydrogen, an amino acid, or (C₁-C₆)alkyl; wherein
any alkyl can be optionally substituted with one or more oxo, hydroxy, amino, phenyl, or carboxy
any alky can be optionally interrupted with one or more oxy or thio;
any phenyl can be optionally substituted with one or more hydroxy or carboxy.

20. The composition of claim 17 wherein R₁ is —O—Y and Y is hydrogen, 3-carboxypropanoyl, 4-carboxybutanoyl, or 2-amino-2-methylbutanoyl.

21. The composition of claim 17 wherein R₂ is hydrogen.

22. The composition of claim 17 wherein R₃ is hydrogen.

23. The composition of claim 17 wherein R₄ is methyl.

24. The composition of claim 17 wherein R₅ is methyl.

25. The composition of claim 17 wherein R₆ is hydrogen and the bond between carbons 12 and 13 is a single bond.

26. The composition of claim 17 wherein R₇ is hydrogen.

27. The composition of claim 17 wherein R₈ and R₁₁ together are —O—CH₂—.

28. The composition of claim 17 wherein R₉ is methyl.

29. The composition of claim 17 wherein R₁₀ is methyl.

30. The composition of claim 17 wherein
R₁ is —O—Y and Y is hydrogen, an amino acid, or (C₁-C₆)alkyl; wherein
the alkyl of Y can be optionally substituted with one or more oxo, hydroxy, amino, carboxy, or phenyl optionally substituted with one or more hydroxy or carboxy;
and can be optionally interrupted with one or more oxy or thio;
R₂ is hydrogen;
R₃ is hydrogen and the bond between carbons 1 and 2 is a single bond;
R₄ and R₅ are each methyl;
R₆ is hydrogen and the bond between carbons 12 and 13 is a single bond;
R₇ is hydrogen
R₈ and R₁₁ together are —O—CH₂—; and
R₉ and R₁₀ are each methyl.

31. The composition of claim 17 wherein the triterpene is 3-β-acetoxy-19αH-19,28 lactone oleanan; allobetulin; allobetulin-3-succinate; allobetulin-3-glycine; allobetulin lactone; allobetulin lactone-3-acetate; allobetulin lactone-3-phosphate; allobetulin-3-L-alanine; allobetulin-3-L-valine; allobetulin-3-L-proline; allobetulin-3-succinate; allobetulin-3-diglycolate; allobetulin-3-phthalate; allobetulin-3-methylenamine; allobetulin-3-ethanolamine; allobetulin-3-glycolate; allobetulin-3-glutarate; allobetulin-28-glutarate; allobetulin-3-methylamine HCl; allobetulin-3-phosphate; allobetulin-3-(polyethylene glycol)-COOH (Mw=674); allobetulon; allobetulon lactone 1-ene-2-ol; allobetulon lactone-1-en-2-succinate; allobetulon-1-ene-2-ol; allobetulon-1-ene-2-diglycolate; 3-allobetulon-1-ene-2-succinate; allobetulin-3-(poly(ethylene glycol)bis (carboxymethyl ester); or 3-allobetulon-1-ene-2-diglycolate.

32. The composition of claim 1 wherein the triterpene is a quaternary ammonium salt of a triterepene.

33. The composition of claim 1 wherein the triterpene is a compound of formula (III): 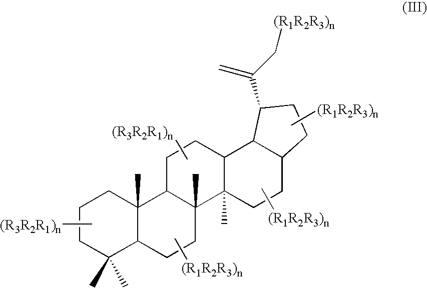
wherein
each R₁ is independently absent, oxy, thio, or imino;
each R₂ is independently absent or alkylene;
each R₃ is independently hydrogen, N⁺-containing heteroaryl, N⁺-containing heterocycle, or —N⁺RₐR_{b}R_{c}; provided at least one R₃ is N⁺-containing heteroaryl, N⁺-containing heterocycle, or —N⁺RₐR_{b}R_{c};
wherein Rₐ, R_{b}, and R_{c} are each independently (C₁-C₂₄)alkyl, aryl, arylalkyl, heteroarylalkly, heterocycle, or heterocylealkyl;
wherein each n is independently 0-4, provided at least one n is not 0;
wherein any heteroaryl, heterocycle, or Rₐ, R_{b}, or R_{c} of R₃ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;
wherein any cycloalkylalkyl can optionally be substituted on carbon with one or more hydroxyl, N⁺-containing heteroaryl, N⁺-containing heterocycle, or —N⁺RₐR_{b}R_{c} N⁺-containing heteroarylalkyloxy, N⁺-containing heterocyclealkyloxy, or —N⁺RₐR_{b}R_{c}oxy;
wherein R_{d} and Rₑ are each independently hydrogen or alkyl;
wherein any alkyl or alkylene of R₃ can optionally be substituted on carbon with one or more oxo, hydroxy, halo, nitro, cyano, (C₁-C₆)alkoxy, trifluoromethyl, —COOR_{d}, or —NR_{d}Rₑ, and optionally interrupted on carbon with one or more oxy, imino, or thio, and is optionally partially unsaturated;
or an acceptable salt thereof.

34. The composition of claim 1 wherein the triterpene is a compound of formula (IV): 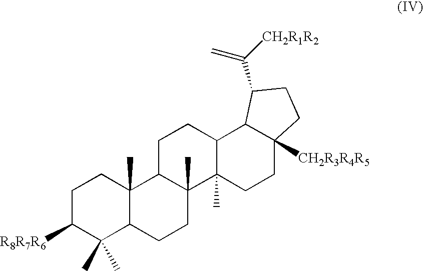
wherein
R₁, R₄, and R₇ are each independently absent or alkylene;
R₃ and R₆ are each independently absent, oxy, thio, or imino;
R₂, R₅, and R₈ are each independently hydrogen, N⁺-containing heteroaryl, N⁺-containing heterocycle, or —N⁺RₐR_{b}R_{c}; provided at least one of R₂, R₅, and R₈ is N⁺-containing heteroaryl, N⁺-containing heterocycle, or —N+RₐR_{b}R_{c};
wherein Rₐ, R_{b}, and R_{c} are each independently (C₁-C₂₄)alkyl, aryl, arylalkyl, heteroarylalkly, heterocycle, or heterocylealkyl;
wherein any heteroaryl, heterocycle, Rₐ, R_{b}, or R_{c} of R₂, R₅, and R₈ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;
wherein any cycloalkylalkyl can optionally be substituted on carbon with one or more hydroxyl, N⁺-containing heteroaryl, N⁺-containing heterocycle, —N⁺RₐR_{b}R_{c}, N⁺-containing heteroarylalkyloxy, N⁺-containing heterocyclealkyloxy, or —N⁺RₐR_{b}R_{c}oxy;
wherein R_{d} and Rₑ are each independently hydrogen or alkyl;
wherein any alkyl or alkylene of R₁, R₂, R₄, R₅, R₇, or R₈ can be optionally substituted on carbon with one or more oxo, hydroxy, halo, nitro, cyano, (C₁-C₆)alkoxy, trifluoromethyl, —COOR_{d}, or —NR_{d}Rₑ, and optionally interrupted on carbon with one or more oxy, imino, or thio, and is optionally partially unsaturated;
or an acceptable salt thereof.

35. The composition of claim 34 wherein R₂, R₅, and R₈ are each independently absent, hydroxyl, N-diazabicyclo[2.2.2]octyl, N-pyridinium, N-alkyl-N-piperidino, N-alkyl-N-morpholino, N-azabicyclo[2.2.2]octyl, or —NRₐR_{b}R_{c}; provided at least one of R₂, R₅, and R₈ is N⁺-containing heteroaryl, N⁺-containing heterocycle, or —N⁺RₐR_{b}R_{c};
wherein N-diazabicyclo[2.2.2]octyl; N-pyridinium;
N-alkyl-N-piperidino; N-alkyl-N-morpholino; and N-azabicyclo[2.2.2]octyl can optionally be substituted on one or more suitable carbon atoms with one or more oxo, hydroxy, mercapto, alkyl, hydroxyalkyl, halo, nitro, cyano, (C₁-C₆)alkoxy, —COOR_{d}, or —NR_{d}Rₑ;
wherein any alkyl or alkylene of R₁, R₂, R₄, R₅, R₇, or R₈ can optionally be substituted with one or more oxo or —NR_{d}Rₑ, and optionally interrupted with one or more oxy, imino, or thio, and can optionally be partially unsaturated.

36. The composition of claim 34 wherein R₁ is absent and R₂ is hydrogen, N-diazabicyclo[2.2.2]octyl, or N-dimethylamino-N-pyridinium.

37. The composition of claim 34 wherein R₃ and R₄ are absent, and R₅ is hydrogen.

38. The composition of claim 34 wherein
R₃ is oxy;
R₄ is absent or (C₁-C₅)alkylenecarbonyl; and
R₅ is hydrogen, N-diazabicyclo[2.2.2]octyl; 4-dimethylamino-N-pyridinium; 4-hydroxybutyl-N-diazabicyclo[2.2.2]octyl; 4-benzyl-N-diazabicyclo[2.2.2]octyl; tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; or tetradecyldimethylammonium.

39. The composition of claim 34 wherein
R₆ is oxy;
R₇ is absent or (C₁-C₅)alkylenecarbonyl; and
R₈ is hydrogen, N-diazabicyclo[2.2.2]octyl; 4-dimethylamino-N-pyridinium; N′-(4-hydroxybutyl)-N-diazabicyclo[2.2.2]octyl; N′-benzyl-N-diazabicyclo[2.2.2]octyl; N,N,N′,N′-tetramethylethylenediamine-N-yl; N′-benzyl-N,N,N′,N′-tetramethylethylenediamine-N-yl; N-pyridinium; 4-hydroxymethyl-N-pyridinium; 2,4-dimethyl-N-pyridinium; 3,5-dimethyl-N-pyridinium; octyldimethylammonium; tetradecyldimethylammonium; 2-methyl-N-pyridinium; 4-hydroxy-N-methyl-N-piperidinium; or N-methyl-N-morpholino.

40. The composition of claim 1 wherein the triterpene is
lup-20(29)-ene-3,28-bis-(N-pyridiniumacetate);
lup-20(29)-ene-3-[N-(4-oxybutyl)-1,4-diazabicyclo[2.2.2]octyl-N′-acetate];
lup-20(29)-ene-3,28-bis[N-(1,4-diazabicyclo[2.2.2]octyl)acetate];
lup-20(29)-ene-3,28-bis[N-(N′-benzyldiazabicyclo[2.2.2]octyl)acetate);
lup-20(29)-ene-3,28-bis[N-(N′-(4-oxybutyl)diazabicyclo[2.2.2]octyl)acetate];
lup-20(29)-ene-3-[N-(1,4-diazabicyclo[2.2.2]octyl)acetate];
lup-20 (29)-ene-3,28-bis[(tetramethylethylenediamine-N-yl)acetate];
lup-20(29)-ene-3,28-bis[(N′-benzyl-N,N,N′,N′-tetramethylethylenediamine-N-yl)acetate];
lup-20(29)-ene-3-[N-(N′-(benzyl)diazabicyclo[2.2.2]octyl)acetate];
bis(N,N′-pyridinium-2-ethyl)lup-20(29)-ene-3,28-dicarbamate;
1-(3,28-(diacetoxy)lup-20(29)-ene-30-yl)-4-(dimethylamino)pyridinium;
lup-20(29)-ene-3,28-bis(N-pyridinium-2-propionate);
lup-20(29)-ene-3,28-bis(N-pyridinium-3-propionate);
lup-20(29)-ene-3,28-bis(N-pyridinium-4-butyrate);
lup-20(29)-ene-3,28-bis(N-pyridinium-4-butyrate);
lup-20(29)-ene-3,28-bis(N-pyridinium-2-butyrate);
1-[3,28-(diacetoxy)lup-20(29)-ene-30-yl]-1,4-diazabicyclo[2.2.2]octyl;
3,28-bis[3-(1-piperidinyl)propanoyloxy]lup-20(29)-ene;
1-(3,28-dihydroxylup-20(29)ene-30-yl)-4-(dimethylamino)pyridinium;
lup-20(29)-ene-3,28-bis[N-(4-dimethylaminopyridinium)-2-propionate];
lup-20(29)-ene-3,28-bis[N-(1,4-diazabicyclo[2.2.2]octyl)-2-propionate];
1-(lup-20(29)-ene-30-yl)-1,4-diazabicyclo[2.2.2]octane;
1-(3,28-dihydroxylup-20(29)-ene-30-yl)-pyridinium;
lup-20(29)-ene-3,28-bis[N-(1,4-diazabicyclo[2.2.2]octyl)-4-butyrate];
1-(3,28-dihydroxylup-20 (29)-ene-30-yl)-[N-3-(hydroxymethyl)pyridinium];
1-(3,28-dihydroxylup-20(29)-ene-30-yl)-[N-(3,5-dimethylpyridinium)];
bis(N-(1,4-diazabicyclo[2.2.2]octyl)-2-ethyl]-lup-20(29)ene-3,28-dicarbamate;
lup-20(29)-ene-3,28-bis[N-(3-oxymethylpyridinium)acetate];
lup-20(29)-ene-3,28-bis[N-(2-oxymethylpyridinium)acetate];
lup-20(29)-ene-3,28-bis[N-(2-methylureapyridinium)acetate];
lup-20(29)-ene-3-[N-(2-oxymethylpyridinium)acetate];
lup-20(29)-ene-3,28-bis[N-(N-methylmorpholino)acetate];
lup-20(29)-ene-3,28-bis[N-(4-hydroxyl-N-methylpiperidino)acetate];
lup-20(29)-ene-3-[N-(3-ureamethylpyridinium)acetate];
lup-20(29)-ene-3-(N-pyridiniumacetate);
lup-20(29)-ene-3,28-bis[N-(1,4-diazabicyclo[2.2.2]octyl)-2-butyrate];
lup-20(29)-ene-3,28-bis[N-(4-dimethylpyridinium)-2-butyrate];
lup-20(29)-ene-3,28-bis[N-(4-dimethylaminopyridinium)-4-butyrate];
lup-20(29)-ene-3,28-bis[N-(4-dimethylaminopyridinium)-3-propionate];
1-(3,28-dihydroxylup-20(29)-ene-30-yl)-4-(hydroxymethyl)pyridinium;
1-(3,28-dihydroxylup-20(29)-ene-30-yl)-3-hydroxy-1-azabicyclo[2.2.2]octane;
lup-20(29)-ene-3,28-bis[N-(2,4-dimethylpyridinium)acetate];
lup-20(29)-ene-3,28-bis[N-(3,5-dimethylpyridinium)acetate];
lup-20(29)-ene-3,28-bis[N-(4-dimethylaminopyridinium)acetate];
lup-20(29)-ene-3-[N-(2-methylpyridinium)acetate];
lup-20(29)ene-3-[N-(2,4-dimethylpyridinium)acetate];
lup-20(29)-ene-3-[N-(4-hydroxy-N-methylpiperidino)acetate];
lup-20(29)-ene-3-[N-(N-methylmorpholino)acetate];
lup-20(29)-ene-3-[N-(3,5-dimethylpyridinium)acetate];
lup-20(29)-ene-3-[N-(4-dimethylaminopyridinium)acetate];
lup-20(29)-ene-3,28-bis(octyldimethylammoniumacetate);
lup-20(29)-ene-3-octyldimethylammoniumacetate;
lup-20(29)-ene-3,28-bis(tetradecyldimethylammoniumacetate);
lup-20(29)-ene-3-tetradecyldimethylammoniumacetate;
N,N,N′,N′-tetramethylethylenediamine-N,N′-bis-[lup-20(29)-ene-3-acetate];
1-[(lup-20(29)-en-3-yl)oxycarbonylmethyl]-4-aza-1-azonia-bicyclo[2.2.2]octane;
1-[(lup-20(29)-en-3-yl)oxycarbonylmethyl]trimethylammonium; or
1-[(lup-20(29)-en-3-yl)oxycarbonylmethyl]pyridinium.

41. The composition of claim 1 wherein the triterpene is a compound of formula (V): 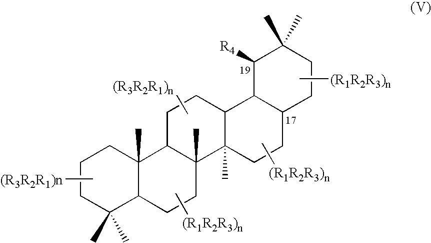
wherein
each R₁ is independently absent, oxy, thio, or imino;
each R₂ is independently absent or alkylene;
each R₃ is independently hydrogen, N⁺-containing heteroaryl, N⁺-containing heterocycle, or —N⁺RₐR_{b}R_{c};
provided at least one R₃ is N⁺-containing heteroaryl, N⁺-containing heterocycle, or —N⁺RₐR_{b}R_{c};
R₄ is hydrogen, alkyl, or hydroxyalkyl;
or R₄ together with one R₁R₂R₃ forms a —OCH₂— bridging carbons 19 and 17;
wherein Rₐ, R_{b}, and R_{c} are each independently (C₁-C₂₄)alkyl, aryl, arylalkyl, heteroarylalkly, heterocycle, or heterocylealkyl;
wherein each n is independently 0-4, provided at least one n is not 0;
wherein any heteroaryl, heterocycle, or Rₐ, R_{b}, or R_{c} of R₃ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;
wherein any cycloalkylalkyl can optionally be substituted on carbon with one or more hydroxyl, N⁺-containing heteroaryl, N⁺-containing heterocycle, —N⁺RₐR_{b}R_{c}, N⁺-containing heteroarylalkyloxy, N⁺-containing heterocyclealkyloxy, or —N⁺RₐR_{b}R_{c}oxy;
wherein R_{d} and Rₑ are each independently hydrogen or alkyl;
wherein any alkyl or alkylene of R₃ can optionally be substituted on carbon with one or more oxo, hydroxy, halo, nitro, cyano, (C₁-C₆)alkoxy, trifluoromethyl, —COOR_{d}, or —NR_{d}Rₑ, and optionally interrupted on carbon with one or more oxy, imino, or thio, and is optionally partially unsaturated or an acceptable salt thereof.

42. The composition of claim 1 wherein the triterpene is a compound of formula (VI) 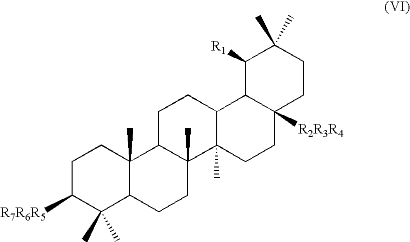
wherein
R₁ is hydrogen, alkyl, or hydroxyalkyl,
R₂ is oxymethylene, thiomethylene, iminomethylene, or methylene;
R₃ and R₆ are each independently absent or alkylene;
R₄ and R₇ are each independently hydrogen, N⁺-containing heteroaryl, N⁺-containing heterocycle, or —NRₐR_{b}R_{c}; provided at least one of R₄ and R₇ is N⁺-containing heteroaryl, N⁺-containing heterocycle, —NRₐR_{b}R_{c}; or R₁, R₂, R₃, and R₄ are together —O—C(═X)—; wherein X is two hydrogens, oxo, or thioxo (═S);
wherein Rₐ, R_{b}, and R_{c} are each independently (C₁-C₂₄)alkyl, aryl, arylalkyl, heteroarylalkyl, heterocycle, or heterocylealkyl;
wherein R₅ is absent, oxy, thio, or imino;
wherein any heteroaryl, heterocycle, or Rₐ, R_{b}, or R_{c} of R₄ and R₇ can optionally be substituted on carbon with one or more alkyl, hydroxyalkyl, arylalkyl, heteroarylalkyl, aryl, heterocycle, heterocyclealkyl, oxo, hydroxy, halo, nitro, cyano, (C₁-C₆)alkoxy, trifluoromethyl, —COOR_{d}, —NR_{d}Rₑ, or cycloalkylalkyl;
wherein any cycloalkylalkyl can optionally be substituted on carbon with one or more hydroxyl, N⁺-containing heteroaryl, N⁺-containing heterocycle, —N+RₐR_{b}R_{c}, N⁺-containing heteroarylalkyloxy, N⁺-containing heterocyclealkyloxy, or —N⁺RₐR_{b}R_{c}oxy;
wherein R_{d} and Rₑ are each independently hydrogen or alkyl;
wherein any alkyl or alkylene of R₃, R₄, R₆, or R₇ can be optionally substituted on carbon with one or more oxo, hydroxy, halo, aryl, nitro, cyano, (C₁-C₆)alkoxy, trifluoromethyl, COOR_{d}, or —NR_{d}Rₑ, and optionally interrupted on carbon with one or more oxy, imino, or thio, and is optionally partially unsaturated;
or an acceptable salt thereof.

43. The composition of claim 42 wherein
R₁ is hydrogen, alkyl, or hydroxyalkyl,
R₂ is oxymethylene, thiomethylene, iminomethylene, or methylene;
R₃ and R₆ are each independently absent or (C₁-C₅)alkylenecarbonyl;
R₄ and R₇ are each independently hydrogen, N-diazabicyclo[2.2.2]octyl; N-pyridinium; N-alkyl-N-piperidino; N-alkyl-N-morpholino; N-azabicyclo[2.2.2]octyl; or NRₐR_{b}R_{c};
or R₁, R₂, R₃, and R₄ are together —O—CH₂—;
wherein N-diazabicyclo[2.2.2]octyl; N-pyridinium; N-alkyl-N-piperidino; N-alkyl-N-morpholino; and N-azabicyclo[2.2.2]octyl can optionally be substituted on carbon with one or more alkyl, hydroxyalkyl, hydroxy, COOR_{d}, or NR_{d}Rₑ;
wherein Rₐ, R_{b}, and R_{c} are each independently aryl or (C₁-C₂₄)alkyl; wherein R_{d} and Rₑ are each independently hydrogen or alkyl;
wherein any alkylene or alkyl can optionally be substituted on carbon with one or more oxo, hydroxy, halo, nitro, cyano, trifluoromethyl, COOR_{d}, or —NR_{d}Rₑ, and optionally interrupted with one or more oxy, imino, or thio, and where any alkyl or alkylene can optionally be partially unsaturated.

44. The composition of claim 42 wherein R₁, R₂, R₃, and R₄ are together —O—CH₂—.

45. The composition of claim 42 wherein R₅ is oxy.

46. The composition of claim 42 wherein R₆ is acetyl.

47. The composition of claim 42 wherein R₇ is N-diazabicyclo[2.2.2]octyl; N-pyridinium; or —N⁺(CH₃)₃.

48. The composition of claim 42 wherein the cation of the compound is
1-[(19,28-epoxy-18-oleanan-3-yl)oxycarbonylmethyl]-4-aza-1-azonia-bicyclo[2.2.2]octane;
[(19,28-epoxy-18-oleanan-3-yl)oxycarbonylmethyl]trimethylammonium; or
1-[(19,28-epoxy-18-oleanan-3-yl)oxycarbonylmethyl]pyridinium.

49. The composition of claim 1 wherein the triterpene is present up to about 30 wt. % of the composition.

50. The composition of claim 1 wherein the triterpene is present up to about 20 wt. % of the composition.

51. The composition of claim 1 wherein the triterpene is present up to about 10 wt. % of the composition.

52. The composition of claim 1 wherein the triterpene is present up to about 5 wt. % of the composition.

53. The composition of claim 1 wherein the essential oil is at least one of ajowan, almond oil, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, camphor, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, coltsfoot, combava petitgrain, coriander, green coriander, cornmint, costus oil, cumin, cypress, davana oil, dill, dill weed, elemi, ephedra, erigeron (fleabane), eucalyptus, eucalyptus citriodora, eucalyptus globulus, lemon eucalyptus, fennel, sweet fennel, fenugreek, fir, fir needle oil, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, menthol, methyl salicylate, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, orange, bitter orange, blood orange, sweet orange, wild West Indian orange, oregano, orris root, concrete orris, osmanthus, palm oil, refined palm oil, palmarosa, paprika, parsley seed, patchouli, Indian patchouli oil, Indonesian patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, sassafras, seabuckthorn berry, sesame oil, sesame seed oil, shea butter, unrefined shea butter, spearmint, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, thymol, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra.

54. The composition of claim 1 wherein the essential oil comprises at least one of menthol, camphor, eucalyptus oil, cedarleaf oil, nutmeg oil, thymol, and turpentine oil.

55. The composition of claim 1 wherein the essential oil is present in a total amount of up to about 90 wt. % of the composition.

56. The composition of claim 1 wherein the essential oil is present in a total amount of up to about 80 wt. % of the composition.

57. The composition of claim 1 wherein the essential oil is present in a total amount of up to about 70 wt. % of the composition.

58. The composition of claim 1 wherein the essential oil is present in a total amount of up to about 60 wt. % of the composition.

59. The composition of claim 1 further comprising water.

60. The composition of claim 1 further comprising at least one of petrolatum, mineral oil, ceresin, and lanolin alcohol.

61. The composition of claim 1 further comprising an absorption enhancer.

62. The composition of claim 61 wherein the absorption enhancer comprises at least one of water, methanol, ethanol, 2-propanol, dimethyl sulfoxide, decylmethyl sulfoxide, tetradecyl methyl sulfoxide, 2-pyrrolidone, N-methyl-2-pyrrolidone, N-(2-hydroxyethyl)pyrrolidone, laurocapram, acetone, dimethyl acetamide, dimethyl formamide, tetrahydrofurfuryl alcohol, docusate sodium, sodium lauryl sulfate, quaternary ammonium salt, lecithin, cephalin, alkylbetamine, monglyceride, diglycxeride, triglyceride, lauryl alcohol, cetyl alcohol, stearyl alcohol, sucrose, sorbitan, polyethylene glycol, urea, and N,N-diethyl-m-toluamide.

63. The composition of claim 1 further comprising a polyhydric alcohol selected from the group of glycerin, ethylene glycol, polyethylene glycol, propylene glycol, triethylene glycol, tetraethylene glycol, sorbitol, and combinations thereof.

64. The composition of claim 1 further comprising a skin protectant selected from the group of aloe, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, mineral oil, ceresin, bisabolol, panthenol, trolamine, white petrolatum, zinc acetate, zinc carbonate zinc oxide, zinc sulfate, and combinations thereof.

65. The composition of claim 1 further comprising an anti-infective agent selected from the group of:
[1R-(1R*, 3S*, 5R*, 6R*, 9R*, 11R*, 15S*, 16R*, 17R*, 18S*, 19E, 21E, 23E, 25E, 27E, 29E, 31E, 33R*, 35S*, 36R*, 37S*)]-33-[(3-Amino-3,6-dideoxy-βD-mannopyranosyl)oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid (Amphotericin B);
5-fluorocytosine (Flucytosine);
2,4-difluoro-α,α¹-bis (1H-1,2,4-triazol-1-ylmethyl) benzyl alcohol)(Fluconazole);
griseofulvin microsize (Griseofulvin);
(E)-N-(6,6-dimethyl-2-hepten-4-ynyl)-N-methyl-1-naphthalenemethanamine hydrochloride)(Terbinafine);
cis-1-acetyl-4-[4-[(2-(2,4-dichlorophenyl)-2-(1H-imadazol-1-ylmethyl)-1,3-dioxolan-4-yl]methoxyl]phenyl]piperazine (Ketoconazole);
(±)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methyoxy]phenyl]-1-piperazinyl]phenyl]-Δ²-1,2,4-triazolin-5-one mixture with (±)-1-[(R*)-sec-butyl]-4-[p-[4-[p-[[(2S*, 4R*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one or (±)-1-[(RS)-sec-butyl]-4-[p-[4-[p-[[(2R, 4S)-2-(2,4-dichlorophenyl)-2-(1H-1,2,4-triazol-1-ylmethyl)-1,3-dioxolan-4-yl]-methoxy]phenyl]-1-piperazinyl]phenyl]-Δ²-1,2,4-triazolin-5-one (Itraconazole);
2-chloro-5-hydroxy-1,3-dimethylbenzene (Chloroxylenol);
griseofulvin ultramicrosize (Griseofulvin);
(E)-N-(6,6,-dimethyl-2-hepten-4-ynyl)-N-methyl-1-naphthalenemanamine hydrochloride (Terbinafine);
6-cyclohexyl-1-hydroxy-4-methyl-2(1H)-pyridinone (Ciclopirox);
N-4-tert-butyl-benzyl-N-methyl-1-naphthalenemethylamine hydrochloride (Butenafine hydrochloride);
nystatin;
(E)-N-(Cinnamyl-N-methyl-1-naphthalenemethylamine hydrochloride (Naftifine hydrochloride);
2′,4′-dichloro-2-imidazol-1-ylacetophenone (Z)-[0-(2,4-dichlorobenzyl)oxime]mononitrate (Oxiconazole nitrate),
6-cyclohexyl-1-hydroxy-4-methyl-2(1H)-pyridone (Ciclopirox);
selenium sulfide;
(±)-1-[4-(p-chlorophenyl)-2-[(2,6-dichlorophenyl)thio]butyl]imidazole mononitrate (Butoconazole nitrate);
([1-(o-chloro-.,.-diphenylbenzyl)imidazole]) (Clotrimazole);
(cis-1-[p-[[2-(2,4-dichlorophenyl)-2-(1H-1,2,4-triazol-1-ylmethyl)-1,3-dioxolan-4-yl]methoxy phenyl]-4-isopropyl-piperazine (Tercanazole);
6-cyclohexyl-1-hydroxy-4-methyl-2(1H)-pyridone (ciclopirox);
and combinations thereof.

66. An anti-fungicidal composition comprising a composition of claim 1 and a fungicidal excipient.

67. The composition of claim 1 which is a cream.

68. The composition of claim 1 which is a gel.

69. The composition of claim 1 which is an ointment.

70. The composition of claim 1 which is a lotion.

71. A therapeutic method for treating a mammal afflicted with a fungal infection comprising administering to the mammal, an effective anti-fungal amount of a composition of claim 1.

72. The method of claim 71 wherein the mammal is a human.

73. The method of claim 71 wherein the fungal infection is caused by a dermatophytic fungus.

74. The method of claim 73 wherein the dermatophytic fungus is Microsporum canis, Microsporum gypseum, Microsporum audouinii, Trichophyton tonsurans, Trichophyton mentagrophytes, Epidermophyton floccosum, Trichophyton rubrum, or Pityrosporum ovale.

75. The method of claim 71 wherein the fungal infection is caused by Candida albicans or Candida guilliermoundi.

76. The method of claim 71 wherein the fungal infection is caused by Blastomyces dermatidis or Cryptococcus neoformans.

77. The method of claim 71 wherein the fungal infection is present on a nail of the mammal, under the nail of the mammal, or a combination thereof.

78. The method of claim 71 wherein the fungal infection is present on a toe-nail of the mammal, under the toe-nail of the mammal, or a combination thereof.

79. The method of claim 71 wherein the fungal infection is present on the scalp of the mammal.

80. The method of claim 71 wherein the fungal infection is present on the vagina of the mammal, in the vagina of the mammal, or a combination thereof.

81. The method of claim 71 wherein the fungal infection is present on a skin surface of the mammal.

82. A method of inhibiting or killing a fungus comprising contacting the fungus with an effective anti-fungal amount of a composition of claim 1.

83. The method of claim 82 wherein the contacting is in vitro.

84. The method of claim 82 wherein the contacting is in vivo.

85. The method of claim 82 wherein the fungal infection is present on plant tissue.

86. The method of claim 82 wherein the fungus is present on turf grass.

87. The method of claim 82 wherein the fungus causes the disease dollar spot or brown patch.

88. The method of claim 85 wherein the plant tissue comprises bark, roots, leaves, flowers, needles, bulbs, berries, rhizomes, rootstocks, stems, seeds, or any combination thereof.
